(19) United States
(12) Patent Application Publication
(10) Pub. No.: US 2014/0202461 A1
(43) Pub. Date: Jul. 24, 2014

(54) OXYGEN COMPRESSOR WITH BOOST STAGE

(71) Applicants: Gerold Goertzen, Homerville, OH (US); Michael R. Nemcek, Avon, OH (US); Martin Fabian, Bay Village, OH (US); William M. Chambers, Grafton, OH (US); Kevin S. Wysocki, Grafton, OH (US)

(72) Inventors: Gerold Goertzen, Homerville, OH (US); Michael R. Nemcek, Avon, OH (US); Martin Fabian, Bay Village, OH (US); William M. Chambers, Grafton, OH (US); Kevin S. Wysocki, Grafton, OH (US)

(73) Assignee: Invacare Corporation, Elyria, OH (US)

(21) Appl. No.: 14/119,192

(22) PCT Filed: May 23, 2012
(86) PCT No.: PCT/US12/39132

§ 371 Date: Nov. 21, 2013

Related U.S. Application Data
(60) Provisional application No. 61/489,392, filed on May 24, 2011.

(51) Int. Cl. A61M 16/10; A61M 16/20; A61M 16/00

(52) U.S. Cl. CPC A61M 16/101; A61M 16/0003; A61M 16/0063; A61M 16/202
USPC 128/202.26

(57) ABSTRACT

An oxygen concentration and compression system includes an oxygen concentrator, a boost stage, a compressor, and a portable container. The boost stage receives oxygen enriched gas at a first pressure from the oxygen concentrator. The boost stage includes a pressure increasing device that increases the pressure of the oxygen enriched gas from the first pressure to a second, controlled pressure. The compressor receives the oxygen enriched gas at the second, controlled pressure and compresses the oxygen enriched gas to a third pressure in the portable container for later use by a patient. A patient outlet provides oxygen enriched gas from the oxygen concentrator or the boost stage for use by a patient.

### RELATED APPLICATION

This application claims the benefit of U.S. provisional application Ser. No. 61/489,392, filed on May 24, 2011, titled “Oxygen Compressor with Boost Stage,” the entire disclosures of which are fully incorporated by reference herein.

### FIELD OF THE INVENTION

The present application relates to the field of gas compressors.

### BACKGROUND

Oxygen has many important medical uses including, for example, assisting patients that have congestive heart failure or other diseases. Supplemental oxygen allows patients to receive more oxygen than is present in the ambient atmosphere. Systems and methods for delivering such oxygen typically include a compressor as a component. U.S. Pat. No. 5,988,165, for example, discloses the use of an inline compressor for this purpose, U.S. Pat. No. 6,923,180 discloses the use of a radial compressor for this purpose, and U.S. Patent Application Publication Pub. No. 2007/0065301 discloses an in-line compressor for this purpose. U.S. Pat. Nos. 5,988,165 and 6,923,180 and U.S. Patent Application Pub. No. 2007/0065301 are incorporated herein by reference in their entirety. In addition, U.S. Patent Application Pub. No. 2011/0038740 is incorporated herein by reference in its entirety.

### SUMMARY

An oxygen concentration and compression system includes an oxygen concentrator, a boost stage, a compressor, and a portable container. The boost stage receives oxygen enriched gas at a first pressure from the oxygen concentrator. The boost stage includes a pressure increasing device that increases the pressure of the oxygen enriched gas from the first pressure to a second, controlled pressure. The compressor receives the oxygen enriched gas at the second, controlled pressure and compresses the oxygen enriched gas to a third pressure in the portable container for later use by a patient. A patient outlet provides oxygen enriched gas from the oxygen concentrator or the boost stage for use by a patient.

### BRIEF DESCRIPTION OF THE DRAWINGS

Further features and advantages of the present invention will become apparent to those of ordinary skill in the art to which the invention pertains from a reading of the following description together with the accompanying drawings, in which:

FIG. 1 is a perspective view of a compressor in accordance with an exemplary embodiment;

FIG. 1A is a second perspective view of the compressor shown in FIG. 1, showing a crankshaft and drive rods of the compressor;

FIG. 1B is a sectional view taken approximately along the plane indicated by lines 1B-1B in FIG. 1;

FIG. 2 is a sectioned perspective view taken along the plane indicated by lines 2-2 in FIG. 1;

FIG. 2A is a sectional view taken along the plane indicated by lines 2-2 in FIG. 1;

FIG. 3 is a sectioned perspective view taken along the plane indicated by lines 3-3 in FIG. 1;

FIG. 3A is a sectional view taken along the plane indicated by lines 3-3 in FIG. 1;

FIG. 4 is a perspective view of an assembly of a crankshaft, drive rods, and pistons;

FIG. 5 is an exploded perspective view of the assembly shown in FIG. 4;

FIG. 6A is a perspective view of a first embodiment of a crankshaft;

FIG. 6B is a sectioned perspective view taken along the plane indicated by lines 6B-6B in FIG. 6A;

FIG. 6C is a view taken along lines 6C-6C in FIG. 6A;

FIG. 6D is a view taken along lines 6D-6D in FIG. 6C;

FIG. 7A is a perspective view of a second embodiment of a crankshaft;

FIG. 7B is a sectioned perspective view taken along the plane indicated by lines 7B-7B in FIG. 7A;

FIG. 7C is a view taken along lines 7C-7C in FIG. 7A;

FIG. 7D is a view taken along lines 7D-7D in FIG. 7C;

FIG. 8A is a sectioned perspective view taken along lines 2-2 with parts removed to illustrate a cylinder and piston assembly;

FIG. 8B is the sectioned perspective view of FIG. 8A with components exploded to illustrate assembly of the piston in the cylinder;

FIG. 9 is a sectional view of a first cylinder head assembly that forms part of the compressor of FIG. 1;

FIG. 10 is a sectional view of a second cylinder head assembly that forms part of the compressor of FIG. 1;

FIG. 11A is a perspective view of a flow path defining spacer;

FIG. 11B is a sectioned perspective view taken along lines 11B-11B in FIG. 11A;

FIG. 12 is a schematic illustration of a first exemplary system of the present invention, including a compressor, for providing oxygen-enriched gas for use by a patient;

FIG. 12A is a schematic illustration that illustrates a system similar to the system shown in FIG. 12 with a boost stage added;

FIG. 12B is a schematic illustration that illustrates a system similar to the system shown in FIG. 12 with a boost stage added;

FIG. 13 is a schematic illustration of a second exemplary system of the present invention, including a compressor, for providing oxygen-enriched gas for use by a patient;

FIG. 13A is a schematic illustration that illustrates a system similar to the system shown in FIG. 13 with a boost stage added;

FIG. 13B is a schematic illustration that illustrates a system similar to the system shown in FIG. 13 with a boost stage added;

FIG. 14 is a schematic illustration of a boost stage for an oxygen concentration and compression system;

FIG. 15 is a schematic illustration of a single stage compressor;

FIG. 16 is an exploded perspective view of a single stage compressor;

FIG. 17A is a perspective view of the single stage compressor shown in FIG. 16;

FIG. 17B is a view taken from the side indicated by lines 17B-17B in FIG. 17A;

FIG. 17C is a view taken from the side indicated by lines 17C-17C in FIG. 17A;

FIG. 17A is a perspective view of the single stage compressor shown in FIG. 16;

FIG. 18 is a schematic illustration of a pressure intensifier;

FIG. 19 is a schematic illustration of a drive arrangement for a pressure increasing device of a boost stage and a compressor of a system for concentrating and compressing oxygen;

FIG. 20 is a perspective view of an exemplary embodiment of a compressor and pressure increasing device having the arrangement illustrated by FIG. 19;

FIG. 21 is an exploded perspective view of the compressor and pressure increasing device of FIG. 20;

FIG. 22 is a schematic illustration of another drive arrangement for a pressure increasing device of a boost stage and a compressor of a system for concentrating and compressing oxygen;

FIG. 23 is a schematic illustration of the boost stage for an oxygen concentration and compression system where the pressure limiting device is a regulator;

FIG. 23A is a schematic illustration of the boost stage for an oxygen concentration and compression system where a differential pressure limiting device is a check valve;

FIG. 24A is a schematic illustration of the boost stage for an oxygen concentration and compression system where the pressure limiting device comprises a valve that is controlled based on input from a pressure sensor; and

FIG. 24B is a schematic illustration of the boost stage for an oxygen concentration and compression system where the pressure limiting device comprises a pressure sensor that is used to control a pressure increasing device.

### DETAILED DESCRIPTION OF PREFERRED EMBODIMENTS

As described herein, when one or more components are described as being connected, joined, affixed, coupled, attached, or otherwise interconnected, such interconnection may be direct as between the components or may be in direct such as through the use of one or more intermediary components. Also as described herein, reference to a “member,” “component,” or “portion” shall not be limited to a single structural member, component, or element but can include an assembly of components, members or elements.

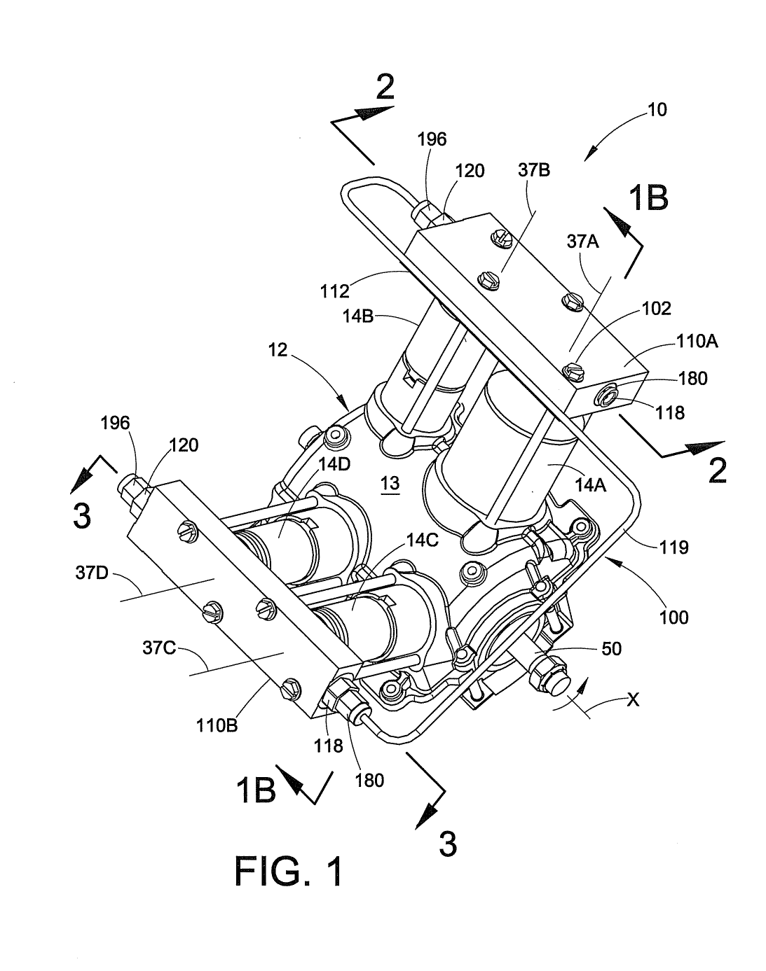

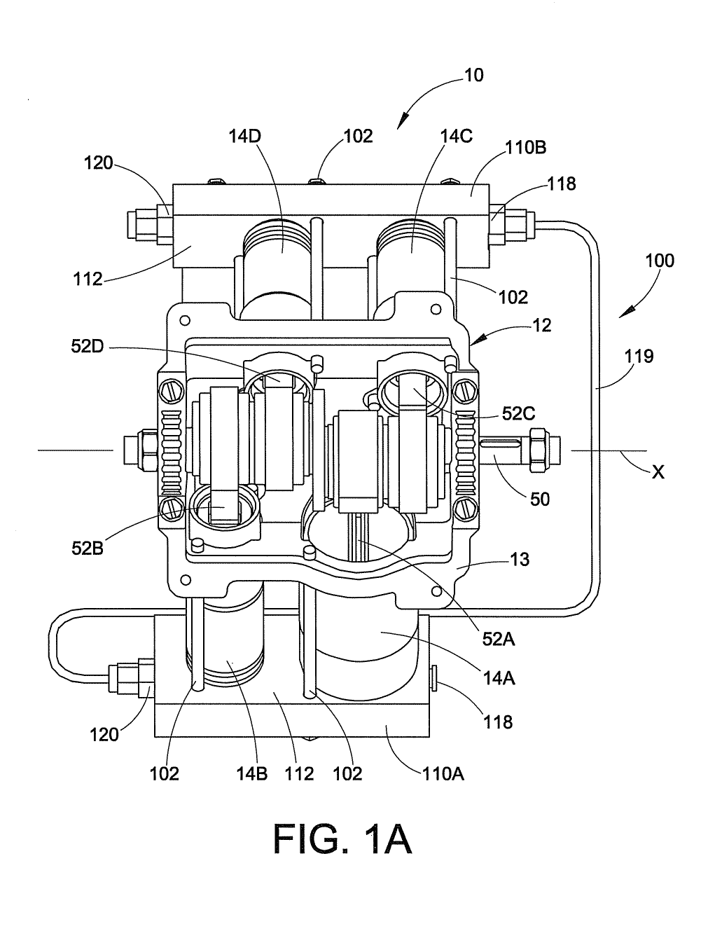

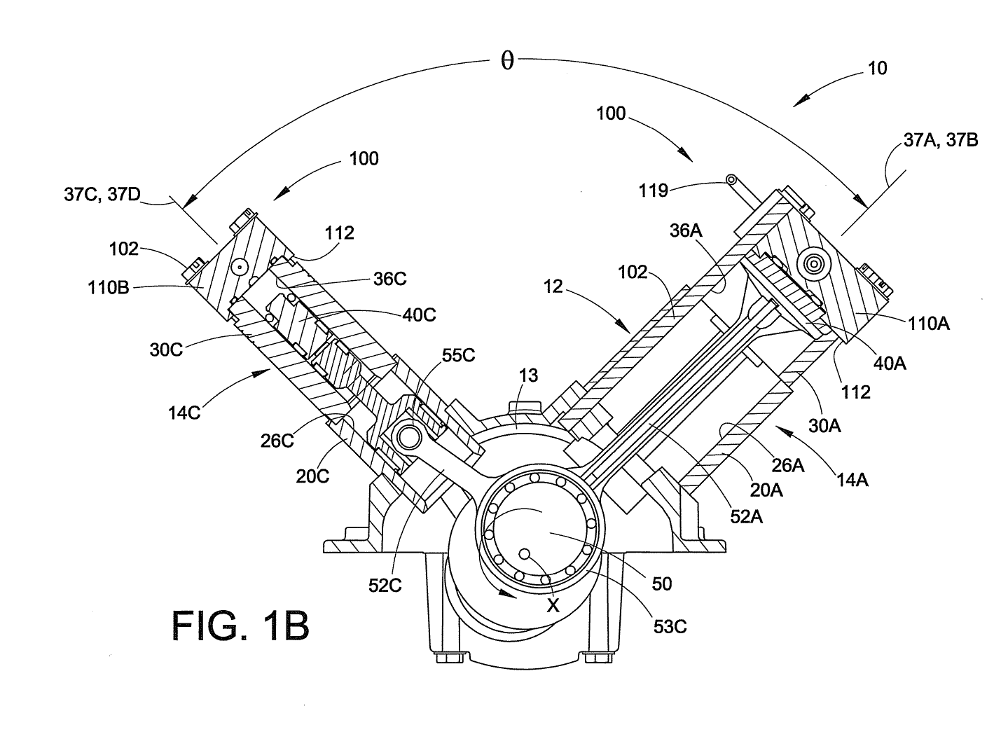

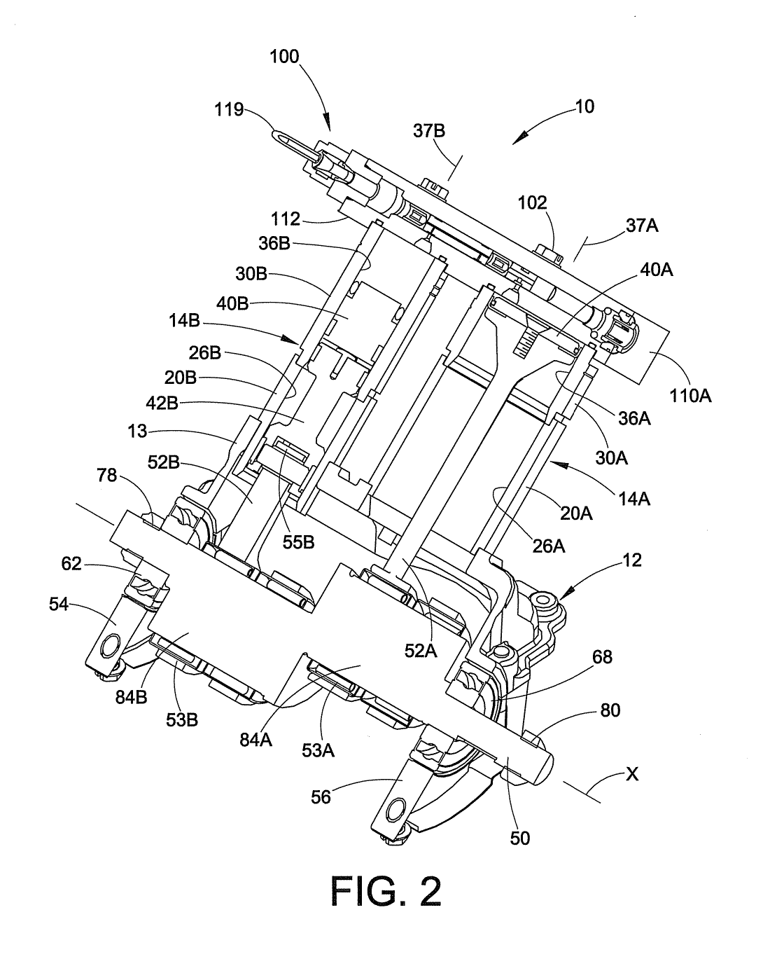

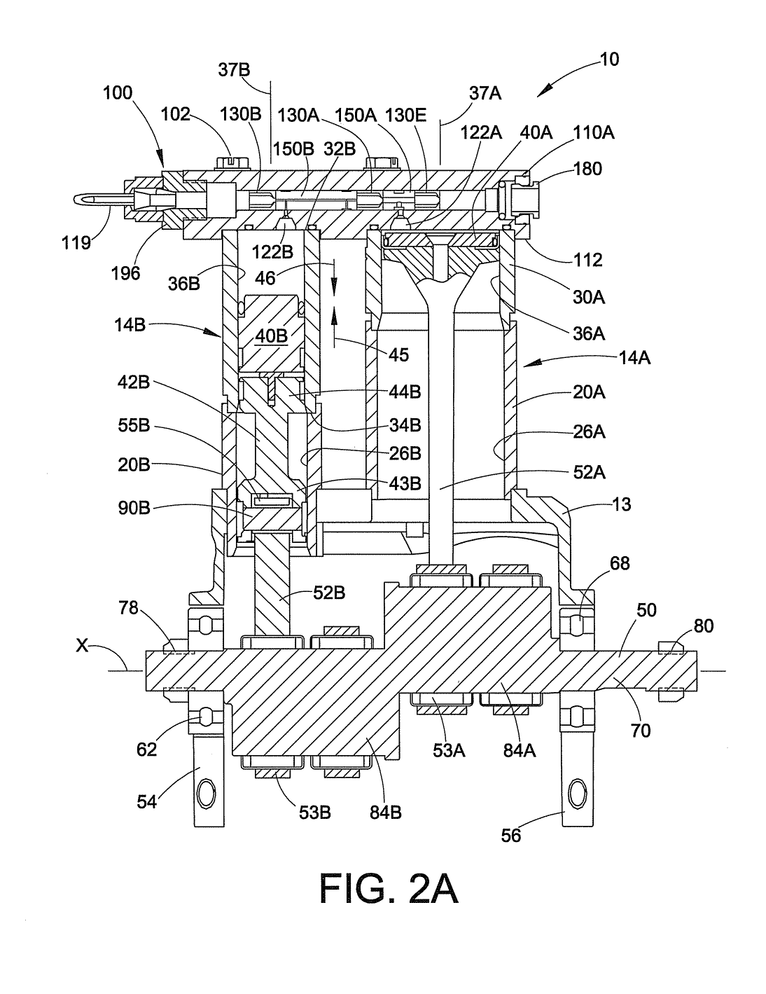

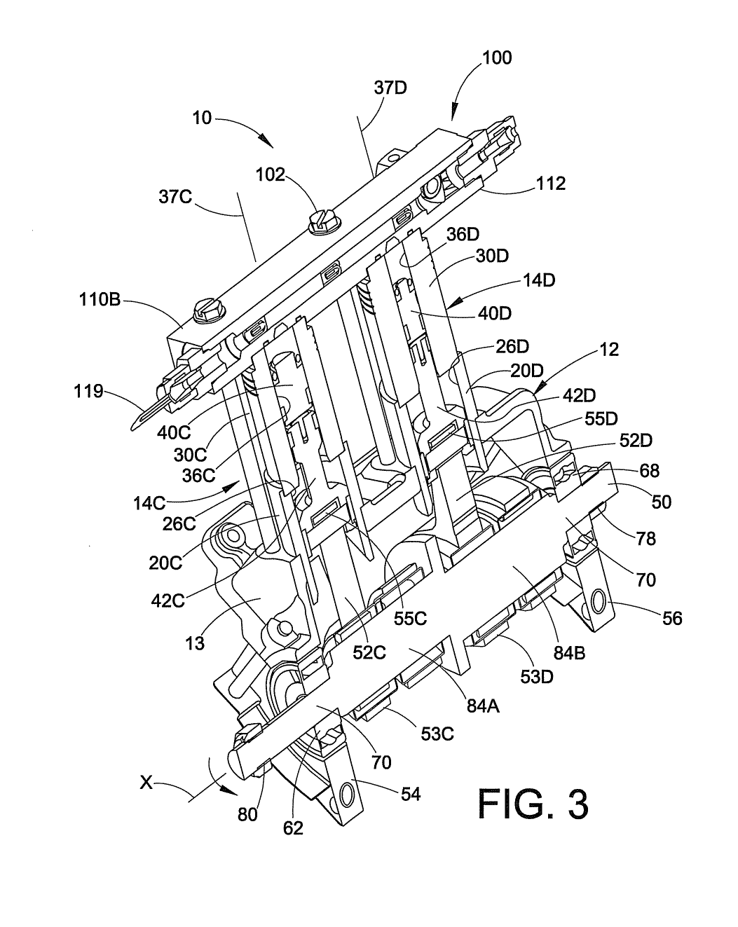

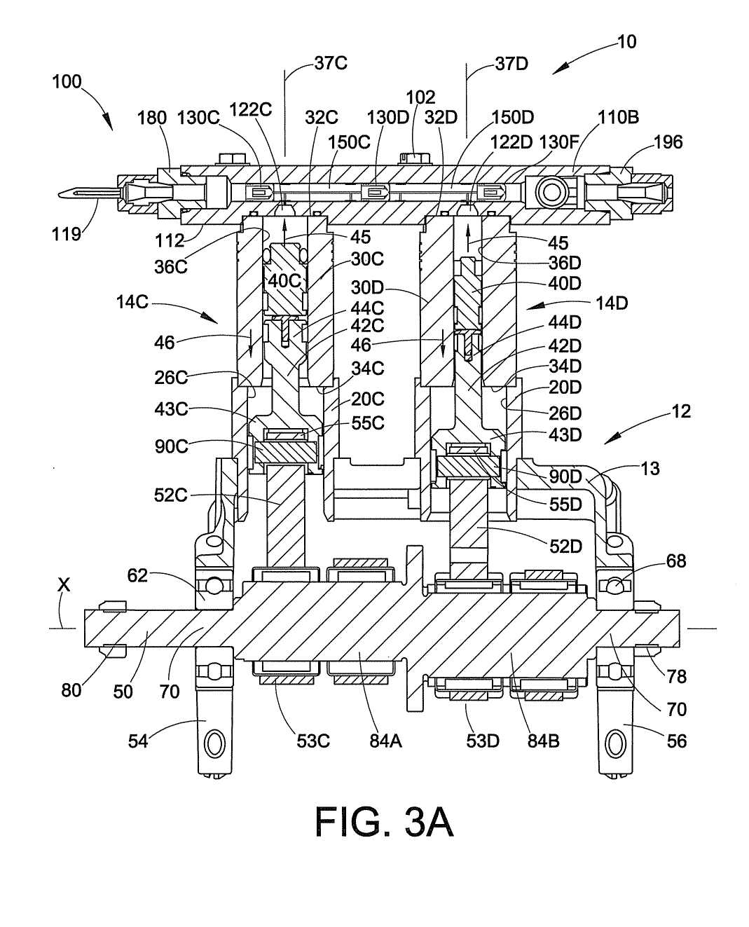

FIG. 1 illustrates an exemplary embodiment of a compressor 10. The compressor 10 includes a cylinder assembly 12 and first and second cylinder heads, 110A, 110B. The cylinder assembly 12 can take a wide variety of different forms. In the example illustrated by FIG. 1, the cylinder assembly includes a base 13, a first sleeve 14A, a second sleeve 14B, a third sleeve 14C, and a fourth sleeve 14D. Referring to FIGS. 2 and 3, in an exemplary embodiment, the first sleeve 14A includes a lower component 20A and an upper component 30A (FIG. 2), the second sleeve 14B includes a lower component 20B and an upper component 30B (FIG. 2), the third sleeve 14C includes a lower component 20C and an upper component 30C (FIG. 3), and the fourth sleeve 14D includes a lower component 20D and an upper component 30D (FIG. 3). The sleeves may take a wide variety of different forms. Any configuration that provides the cylinders can be used. For example, one or more of the cylinders may be formed in only a single component. The first and/or second sleeves and/or the third and fourth sleeves, may be a formed from a single piece or block.

Referring to FIGS. 2 and 3, the lower sleeve components 20A, 20B, 20C, 20D each have an opening 26A-26D. The openings 26A-26D may take a variety of different forms. One or more of the openings 26A-26D may be configured to act as a guide. Further, one or more of the openings 26A-26D may have the same size as one or more of the other openings 26A-26D. The opening 26A is adjacent and inline with the opening 26B and the guide opening 26C is adjacent and inline with the opening 26D in the illustrated embodiment. Referring to 1B, an angle θ between the guide openings 26A, 26B and the guide openings 26C, 26D is approximately 90 degrees in the exemplary embodiment. For example, the angle θ may be and angle in the range between 80 and 100 degrees in one exemplary embodiment, such as an angle between 85 and 95 degrees.

Referring to FIGS. 2 and 3, the upper sleeve components 30A-30D include openings or cylinders 36A-36D. The cylinders 36A-36D may take a variety of different forms. The cylinders 36A-36D are inline with the openings 26A-26D. As such, the angle θ is defined between the cylinders 36A, 36B and the cylinders 36C, 36D. As such, the cylinders 36A-36D are in a substantially “V4” configuration. That is, the central axes 37A, 37B of the cylinders 36A, 36B from a “V” shape with respect to the central axes 37C, 37D of the cylinders 36C, 36D (see FIG. 1B). As can be seen in FIGS. 1, 2, and 3, the central axes 37A-37D are each axially offset from one another in the illustrated embodiment.

Referring to FIGS. 2 and 3, the compressor includes a plurality of pistons 40A-40D that are associated in a one to one relationship with the cylinders 36A-36D. A first piston 40A is located in the first cylinder 36A and is supported for sliding (reciprocating) movement in the first cylinder (FIG. 2). A second piston 40B is located in the second cylinder 36B and is supported for sliding (reciprocating) movement in the second cylinder (FIG. 2). A third piston 40C is located in the third cylinder 36C and is supported for sliding (reciprocating) movement in the third cylinder (FIG. 3). A fourth piston 40D is located in the fourth cylinder 36D and is supported for sliding (reciprocating) movement in the fourth cylinder (FIG. 3).

The cylinders 36A-36D and corresponding pistons 40A-40D are of varying diameters and as a result, the stroke of each piston 40A-40D in its respective cylinder results in a different displacement of gas during the stroke of each piston. The concept of pistons 40A-40D having different strokes from one another may optionally be implemented in the compressor 10. If the strokes of the pistons are different from one another, one or more of the pistons may have the same diameter as one or more other pistons. In the illustrated embodiment, the first cylinder 36A is the largest in diameter, the second cylinder 36B is smaller than the first cylinder, the third cylinder 36C is smaller yet, and the fourth cylinder 36D is the smallest. In other embodiments, the compressor may have more than four cylinders or fewer than four cylinders.

As indicated above, the upper sleeves 30A-30D are in engagement with lower sleeves 20A-20D. The openings 26A-26D in the lower guide sleeves align with the cylinders 36A-36D in the upper cylinder sleeves. The compressor 10 may include one or more guides that are slideably disposed in the openings 26A-26D. Referring to FIGS. 2-4, the compressor includes guides 42B-42D slideably disposed in the openings 26B-26D and a guide is not included in the first opening 26A in the illustrated embodiment. However, guides may be included in all of the openings 26A-26D or any number of guides may be included. The illustrated guides 42B-D are driven by a crankshaft 50 and connecting rods 52B-52D, as described below. The illustrated connecting rods 52B-52D each include a first ring portion 53B-53D and a second ring portion 55B-55D for pivotal connection to the crankshaft 50 and the guides 42B-42D respectively (See FIGS. 2 and 3).

In the illustrated embodiment, no guide is disposed in the opening 26A. The first piston 40A is fixed for movement with the drive or connecting rod 52A. This arrangement is referred to as a “wobble piston,” because fixing the piston 40A to the connecting rod 52A causes some amount of canting or wobbling as the piston 40A moves in the cylinder 36A. Alternatively, the first piston 40A could be pivotally connected to the connecting rod 52A in a conventional manner. In this embodiment, the first piston 40A will slide in the cylinder 36A without significant canting or wobbling. The illustrated connecting or drive rod 52A includes a ring portion 53A for rotatable connection to a crankshaft 50.

Referring to FIG. 2A, the illustrated guide 42B includes a first portion 43B and a second portion 44B. The first portion 43B of the guide 42B is located in the opening 26B and is supported for sliding (reciprocating) movement in the opening. The second portion 44B of the guide 42B is located in the cylinder 36B and is supported for sliding (reciprocating) movement in the cylinder 36B. In the embodiment illustrated by FIGS. 2 and 2A, the second piston 40B is separate from the guide 42B and is not attached to the guide. In this embodiment, during a compression stroke (illustrated by arrow 45 in FIG. 2A), the guide 42B forces the second piston 40B toward the end surface 32B or head end of the cylinder 36B. During a charging stroke (illustrated by arrow 46 in FIG. 2A), gas pressure applied to the cylinder 36B by the first piston 40A forces the second piston 40B toward the end surface 34B or crankshaft end of the cylinder. In an exemplary embodiment, the second piston 40B remains in contact with the second portion 44B of the guide 42B during both the entire compression stroke and the entire charging stroke. In another embodiment, the second piston 40B is fixed or connected for movement with the guide 42B.

Referring to FIG. 3A, the illustrated guide 42C includes a first portion 43C and a second portion 44C. The first portion 43C of the guide 42C is located in the opening 26C and is supported for sliding (reciprocating) movement in the opening. The second portion 44C of the guide 42C is located in the cylinder 36C and is supported for sliding (reciprocating) movement in the cylinder 36C. In the embodiment illustrated by FIG. 3, the third piston 40C is separate from the guide 42C and is not attached to the guide. In this embodiment, during a compression stroke (illustrated by arrow 45 in FIG. 3A), the guide 42C forces the third piston 40C toward the end surface 32C or head end of the cylinder 36C. During a charging stroke (illustrated by arrow 46 in FIG. 3A), gas pressure applied to the cylinder 36C by the second piston 40B forces the third piston 40C toward the end surface 34C or crankshaft end of the cylinder. In an exemplary embodiment, the third piston 40C remains in contact with the second portion 44C of the guide 42C during both the entire compression stroke and the entire charging stroke. In another embodiment, the third piston 40C is fixed or connected for movement with the guide 42C.

Referring to FIG. 3A, the illustrated guide 42D includes a first portion 43D and a second portion 44D. The first portion 43D of the guide 42D is located in the opening 26D and is supported for sliding (reciprocating) movement in the opening. The second portion 44D of the guide 42D is located in the cylinder 36D and is supported for sliding (reciprocating) movement in the cylinder 36D. In the embodiment illustrated by FIG. 3A, the fourth piston 40D is separate from the guide 42D and is not attached to the guide. In this embodiment, during a compression stroke (illustrated by arrow 45 in FIG. 3A), the guide 42D forces the fourth piston 40D toward the end surface 32D or head end of the cylinder 36C. During a charging stroke (illustrated by arrow 46 in FIG. 3A), gas pressure applied to the cylinder 36D by the third piston 40C forces the fourth piston 40D toward the end surface 34D or crankshaft end of the cylinder. In an exemplary embodiment, the fourth piston 40D remains in contact with the second portion 44D of the guide 42D during both the entire compression stroke and the entire charging stroke. In another embodiment, the fourth piston 40D is fixed or connected for movement with the guide 42D.

Referring to FIGS. 2 and 3, crankshaft 50 (described below in detail) is supported for rotation about a crank axis X in first and second bearings 62, 68. The first and second bearings 62, 68 are mounted to the base 13 by first and second and second bearing supports 54 and 56 that are located at either end of the compressor base 13.

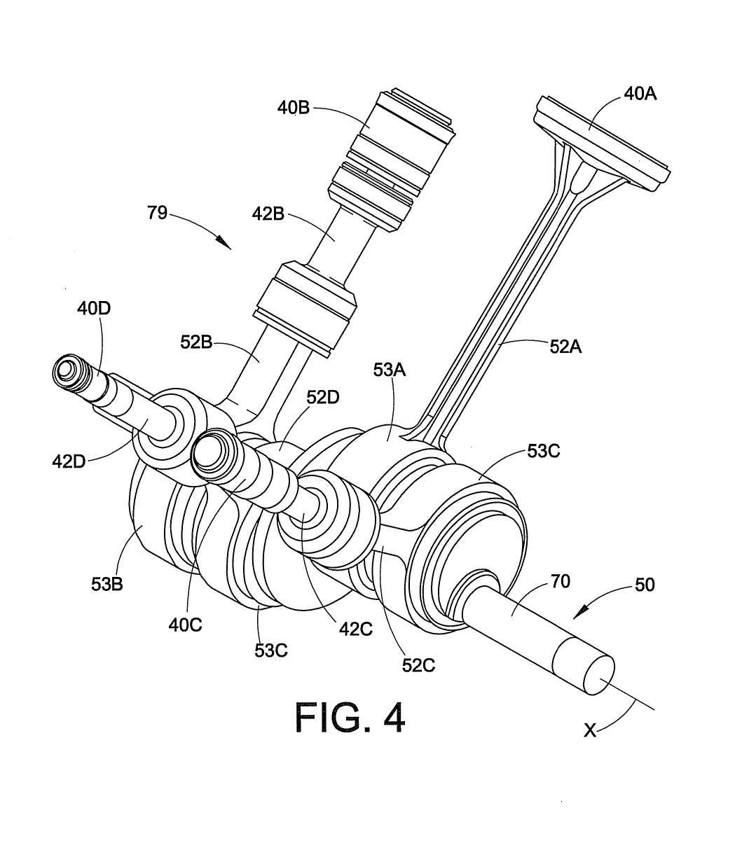

Referring to FIG. 4, the crankshaft 50 forms part of a drive mechanism 79 of the compressor 10 for driving the pistons 40A-40D for movement in the cylinders 36A-36D. The drive mechanism 79 includes the crankshaft 50, the drive or connecting rods 52A-52D, and the guides 42B-42D. However, a wide variety of different drive mechanisms may be used. In other embodiments the crankshaft could be connected to the pistons or coupled to the pistons 40A-40D in other manners, for example with connecting or drive rods but not guides.

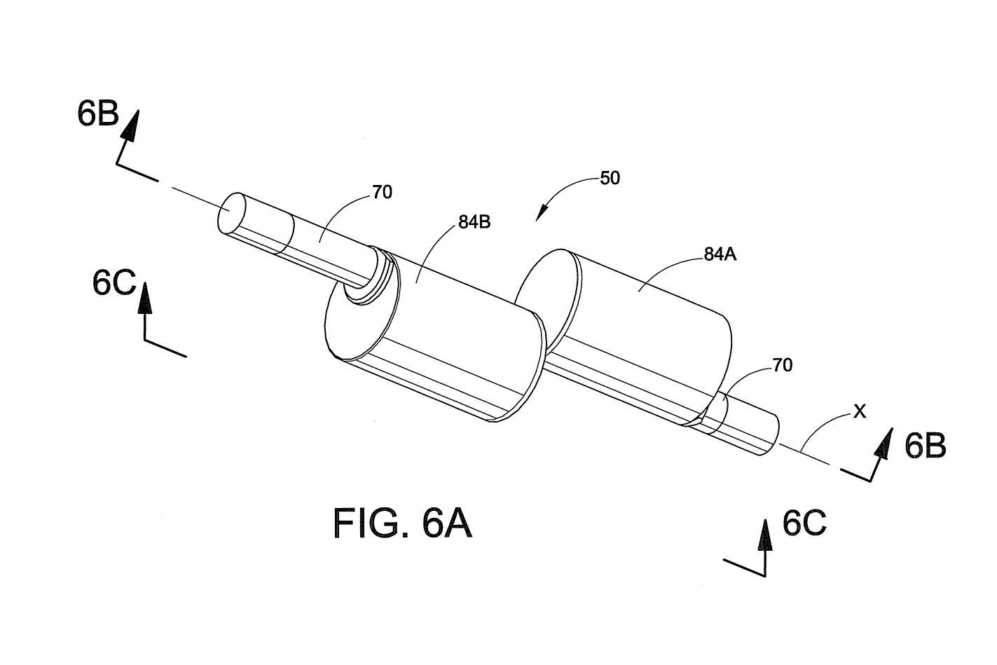

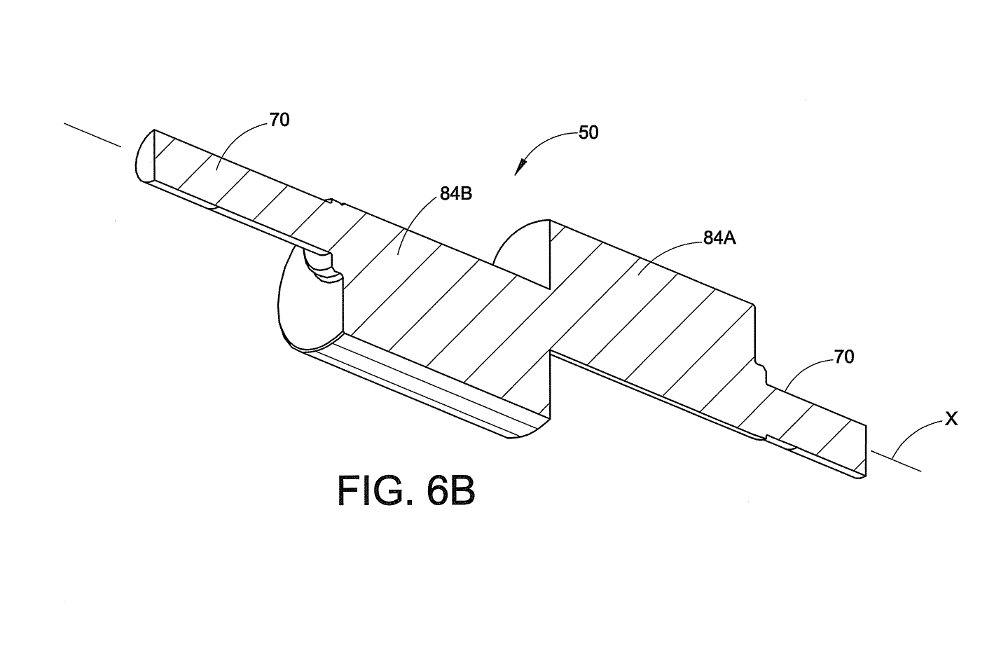

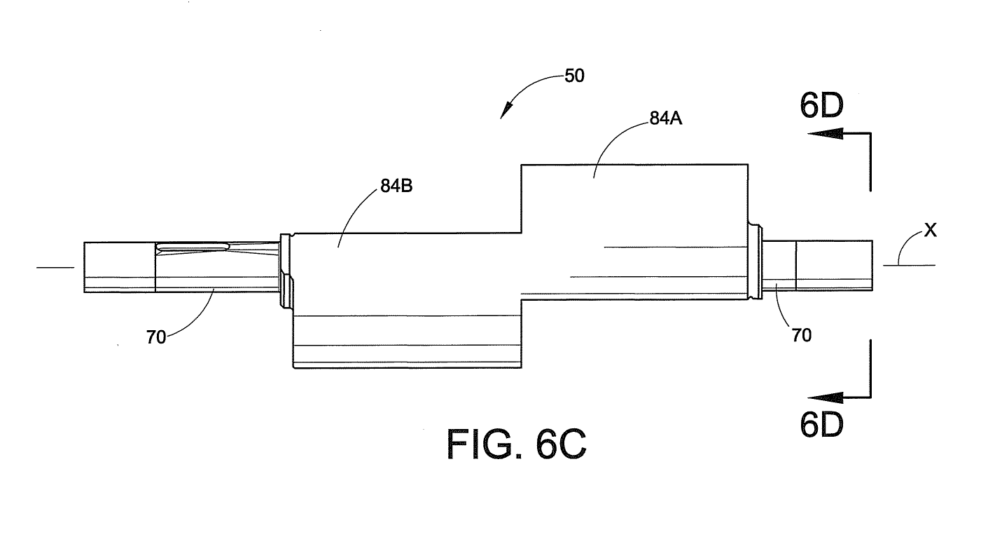

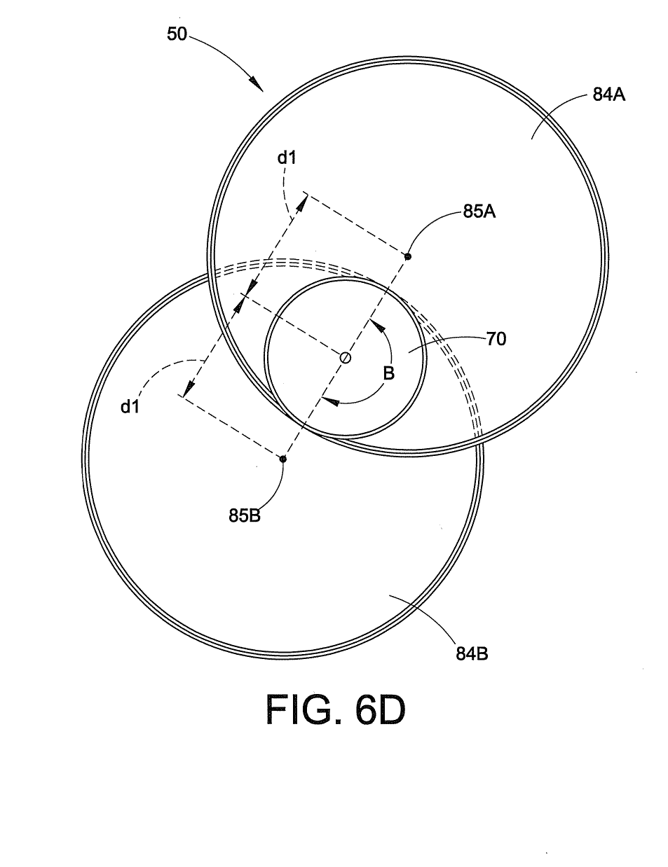

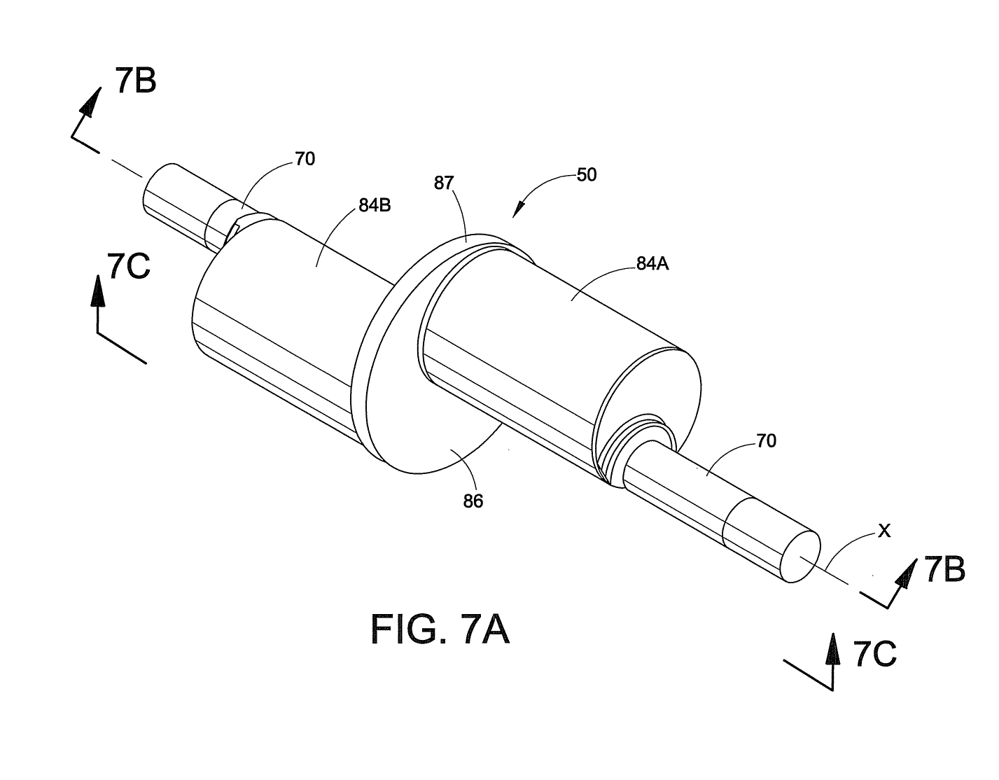

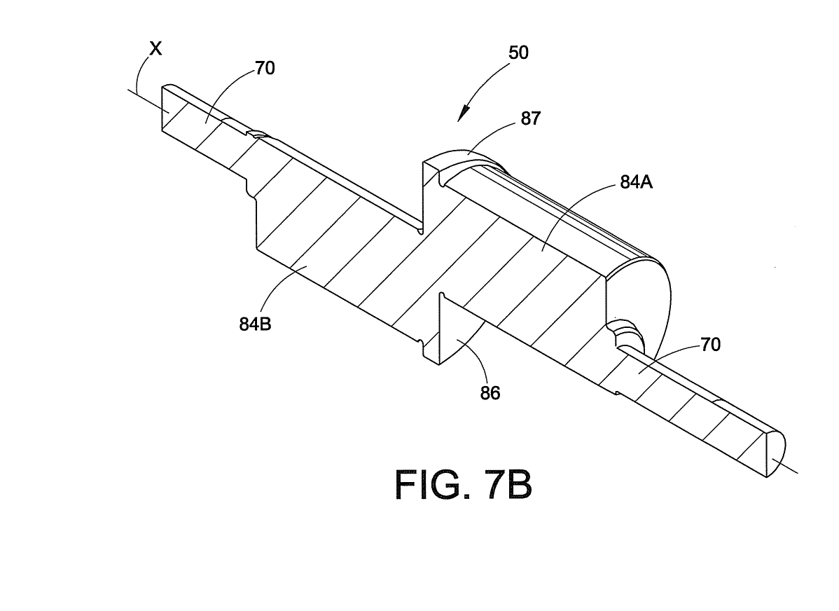

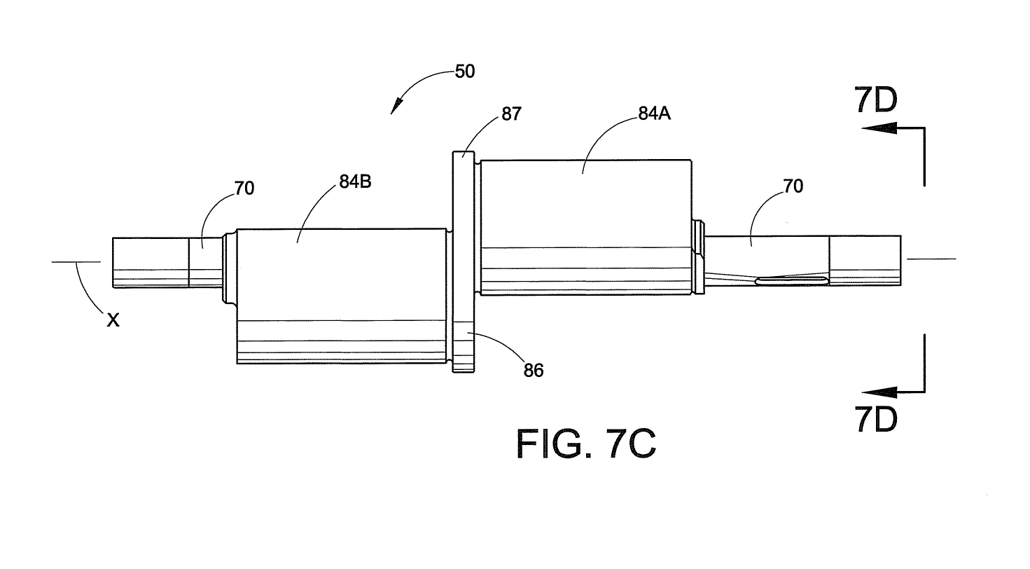

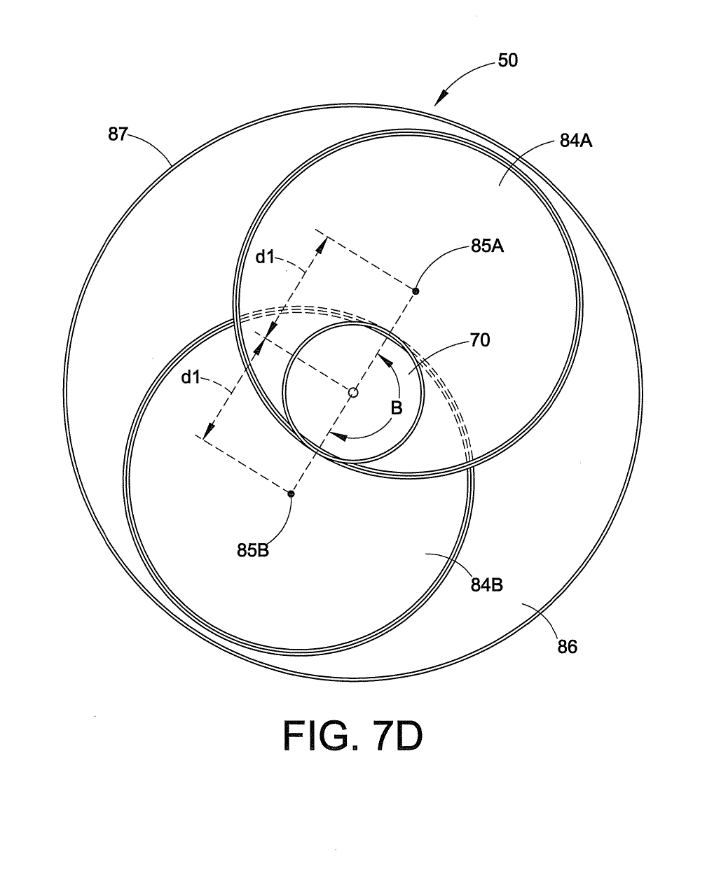

FIGS. 6A-6D and 7A-7D illustrate two embodiments of crankshafts 50. In the embodiments illustrated by FIGS. 6A-6D and 7A-7D the crankshaft 50 is made from a single piece (or welded together to form a single piece). However, the crankshaft 50 may be made from multiple pieces that are assembled together and can be disassembled.

The crankshaft 50 includes a main shaft 70 having a generally cylindrical configuration defined by a cylindrical outer surface centered on a crank axis X of the compressor 10. The crankshaft 50 rotates about the crank axis X during operation of the compressor 10. In the illustrated embodiments, the main shaft 70 has externally threaded opposite end portions 78 and 80. Referring to FIGS. 1-3, the main shaft 70 is received and supported in the first and second bearings 62 and 68.

Referring to FIGS. 6A-6D and 7A-7D, in the illustrated embodiments, the crankshaft 50 also includes first and second circular connecting rod driving bodies 84A, 84B that extend radially outward from and are eccentric to the crank axis X. In the illustrated embodiments, the bodies 84A, 84B are identical to each other, for ease of manufacturing. However, the bodies 84A. 84B may have different sizes, for example such that the body 84A provides a different stroke than body 84B. Referring to FIGS. 6D and 7D, each of the eccentric bodies 84A, 84B has a cylindrical configuration with each cylinder having a central axis 85A, 85B that is parallel to, but spaced apart from the crank axis X. In the illustrated embodiments, the central axis 85A and the central axis 85B are positioned away from the crank axis X by the same distance d1 and an angle β of approximately 180 degrees (See FIG. 6D) is formed between the central axis 85A, the crank axis X, and the central axis 85B. However, the bodies 84A, 84B can be positioned with respect to the crank axis in any manner to achieve desired motions of crank or drive rods 54A-54D that are coupled to the bodies. In the illustrated embodiment, the main shaft portion 70 that is mounted in the bearings 62, 68 has a diameter that is less than a diameter of the circular connecting rod driving bodies 84A, 84B.

Referring to FIG. 4, in an exemplary embodiment the first and second circular connecting rod driving bodies 84A, 84B are the only connecting rod driving bodies of the crankshaft. In this embodiment, each of the connecting rod driving bodies drives two connecting or drive rods 54A-54D as will be described in more detail below. However, any number of connecting rod driving bodies can be included. For example, one connecting rod driving body may be included for each connecting or drive rod. Further, one or more connecting rod driving bodies may drive one connecting or drive rod and one or more connecting rod driving bodies may drive two or more connecting or drive rods.

The connecting rod drive bodies 84A, 84B may take a wide variety of different forms. In the embodiments illustrated by FIGS. 6A-6D and 7A-7D, the connecting rod driving bodies 84A, 84B are each formed as a single continuous cylinder. The illustrated continuous cylinders are integrally formed with the main shaft 70. In another embodiment, the connecting rod driving bodies are two separately formed continuous cylindrical members that are assembled with the main shaft 70. The two separately formed continuous cylindrical members may be identical or may have different sizes to provide different strokes.

In the embodiment illustrated by FIGS. 6A-6D, the first connecting rod driving body 84A abuts the second connecting rod driving body 84B. The first connecting rod driving body 84A may be integrally formed with the second connecting rod driving body 84B, or the connecting rod driving bodies 84A, 84B may be separate pieces that are fixed together. In the example illustrated by FIGS. 6A-6D, the first connecting rod driving body 84A is connected to the second connecting rod driving body 84B only at an area of overlap between the first connecting rod driving body and the second connecting rod driving body.

In the embodiment illustrated by FIGS. 7A-7D, the first connecting rod driving body 84A is connected to the second connecting rod driving body 84D by a circular disk 86 disposed between the first connecting rod driving body 84A and the second connecting rod driving body 84B. The connecting rod driving bodies 84A, 84B may be separate from one another and then fixed to the circular disk 86 or the connecting rod driving body 84A, the circular disk 86, and the connecting rod driving body 84A may be integrally formed. In the embodiment illustrated by FIGS. 7A-7D, the circular disk 86 is centered on the crank axis X. Referring to FIG. 7D, the illustrated circular disk has an outer circumference 87 that is radially outward of the outer circumferences of both of the first and second connecting rod driving bodies 84A, 84B.

As shown in FIGS. 2 and 2A a connecting rod 52A is connected between the first piston 40A and the first eccentric connecting rod driving body 84A and a connecting rod 52B is connected between the guide 42B (which drives the second piston 40B) and the second eccentric connecting rod driving body 84B. In the illustrated embodiment, the ring 53A is disposed around the body 84A to rotatably connect the rod 52A to the body 84A. A bearing may be disposed between the ring 53A and the body 84A. The ring 53B is disposed around the body 84B to rotatably connect the rod 52B to the body 84B. A bearing may be disposed between the ring 53B and the body 84B. A pin 90B extends through the ring portion 55B to pivotally connect the guide 42B the rod 52B.

Referring to FIGS. 3 and 3A, a connecting rod 52C is connected between the guide 42C (which drives the third piston 40C) and the first eccentric connecting rod driving body 84A and a connecting rod 52D is connected between the guide 42D (which drives the fourth piston 40D) and the second eccentric connecting rod driving body 84B. In the illustrated embodiment, the ring 53C is disposed around the body 84A to rotatably connect the rod 52C to the body 84A. A bearing may be disposed between the ring 53C and the body 84A. A pin 90C extends through the ring portion 55C to pivotally connect the guide 42C to the rod 52C. The ring 53D is disposed around the body 84B to rotatably connect the rod 52D to the body 84B. A bearing may be disposed between the ring 53D and the body 84B. A pin 90D extends through the ring 55D to pivotally connect the guide 42D to the rod 52D.

The first eccentric connecting rod driving body 84A drives both the first and third pistons 40A, 40C. Referring to FIG. 1B, due to the “V” configuration of the pistons, the motion of the third piston 40C follows or lags the motion of the first piston 40A by rotation of the crankshaft by the angle of the “V” θ (approximately 90 degrees in the illustrated embodiment). The second eccentric connecting rod driving body 84B drives both the second and fourth pistons 40B, 40D. Due to the angular spacing β of the first and second connecting rod driving bodies 84A, 84B about the crank axis X, the motion of the second piston 40B follows or lags the motion of the first piston 40A by rotation of the crankshaft by the angle of the angular spacing β (approximately 180 degrees in the illustrated embodiment). Due to the “V” configuration of the pistons, the motion of the fourth piston 40D follows or lags the motion of the second piston 40B by rotation of the crankshaft by the angle of the “V” θ (approximately 90 degrees in the illustrated embodiment).

Rotation of the main shaft 70 about the crank axis X results in reciprocating movement of pistons 40A-40D in the cylinders 36A-36D. A drive pulley (not shown) may be located on one of the end portions 78 of the main shaft 70 to facilitate the application of a drive torque to the main shaft 70, to reciprocate the pistons 40A-40D.

As shown in FIG. 1, the compressor 10 includes a cylinder head assembly 100. The cylinder head assembly 100 includes a first cylinder head 110A and a second cylinder head 110B that is fastened to the cylinder assembly 12 with a plurality of fasteners. In the illustrated embodiment, the compressor 10 includes fasteners, such as bolts 102 that extend through holes in the cylinder heads 110A, 110B and are threaded into the base 13. When the bolts 102 are tightened down, the cylinder head 110A is clamped to the first and second sleeves 14A, 14B and the cylinder head 110B is clamped to the third and fourth sleeves 14C, 14D.

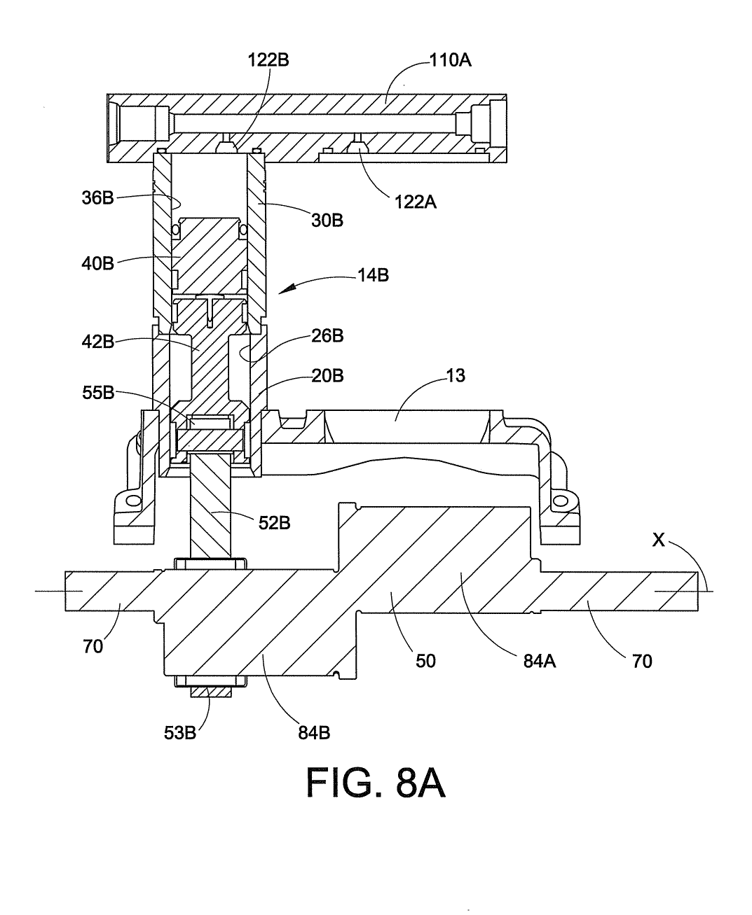

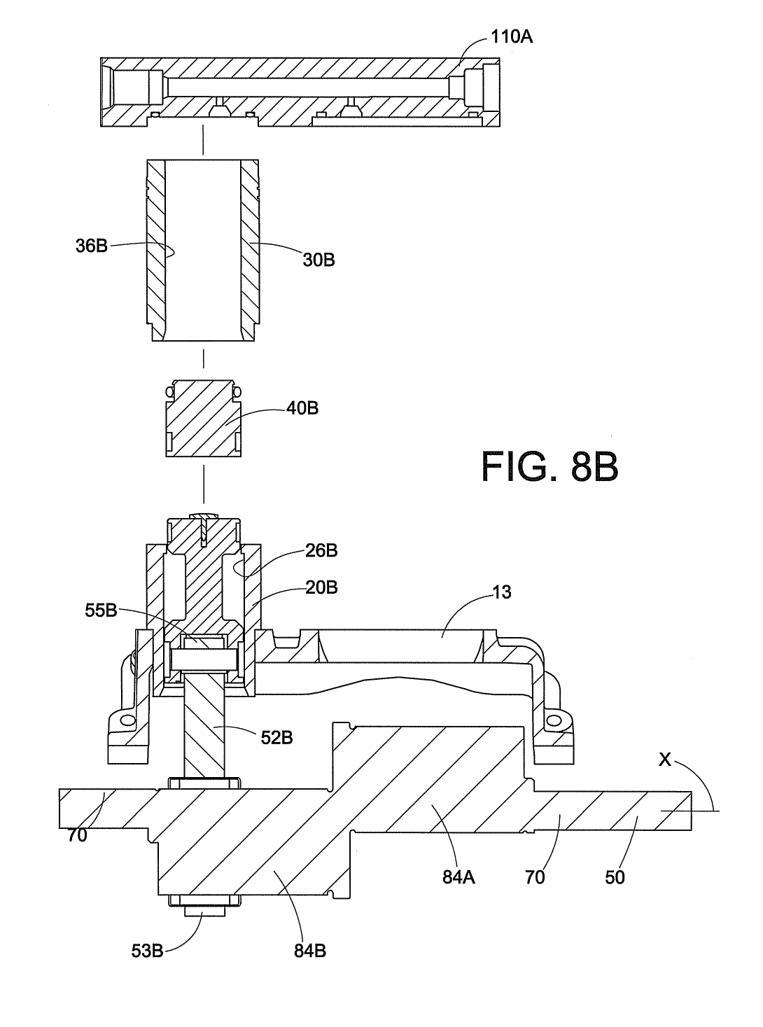

Referring to FIGS. 8A and 8B, for repair or servicing, each of the separate pistons 40B-40D can be removed from the cylinders 36B-36D by removing the fasteners 102 (See FIG. 1) that hold the head 110A and/or 110B down. The second cylinder 36B and piston 40B is illustrated in FIGS. 8A and 8B, but the other pistons and cylinders can be repaired or serviced in the same manner. Once the fasteners 102 are removed, the head 110A, the cylinder 36B, and the piston 40B can be removed and separated as illustrated by FIG. 8B. This arrangement allows the piston 40B and/or cylinder 36B to be replaced or serviced without requiring the drive or connecting rod 52B to be removed from the crankshaft 50.

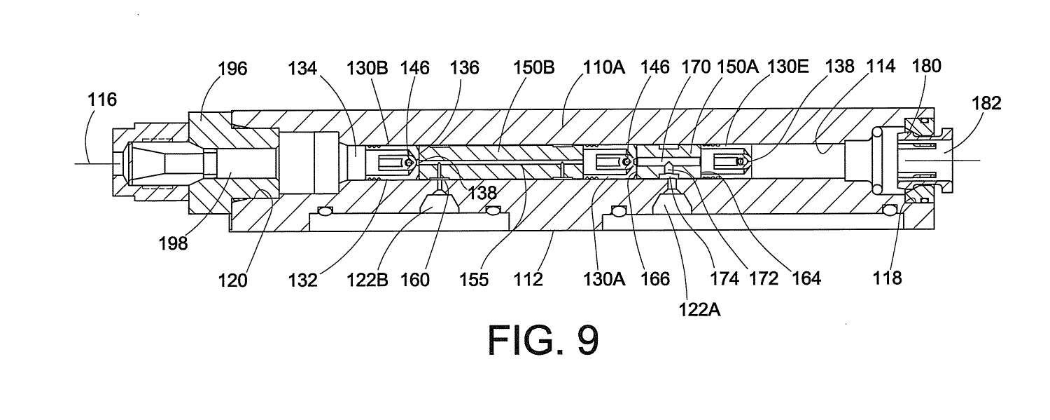

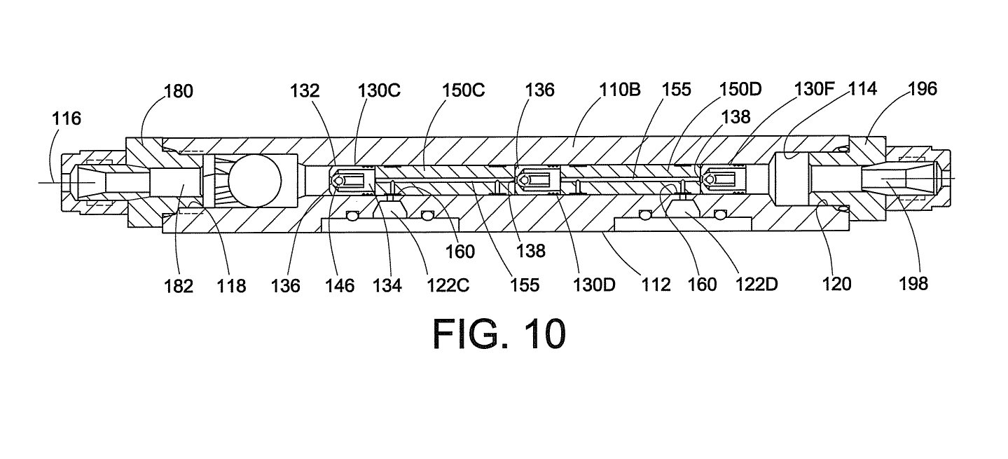

As shown in FIGS. 1, 9 and 10, each cylinder head 110A, 110B is formed as one piece from metal. In the illustrated embodiment, each cylinder head 110A, 110B has a rectangular configuration including a lower side surface 112. Referring to FIGS. 9 and 10, a component chamber 114 extends the length of each cylinder head 110A, 110B. In the illustrated embodiment, the component chambers 114 each have a cylindrical configuration centered on an axis 116. Each component chamber 114 has an inlet end portion 118 and an outlet end portion 120. The inlet end portion 118 of the first cylinder head 110A forms an inlet of the compressor 10. The outlet end portion 120 forms an outlet of the first cylinder head 110A. The inlet end portion 118 of the second cylinder head 110B forms an inlet to the second head 110B. Referring to FIG. 1, a conduit 119 connects the outlet of the first head 110A to the inlet of the second head 110B. The threaded outlet end portion 120 of the second head 110b forms an outlet of the compressor 10.

Referring to FIGS. 9 and 10, the cylinder heads 110a, 110b have a plurality of charging ports 122A-122D that extend between the component chamber 114 and the lower side surface 112. The number of charging ports 122A-122D is equal to the number of cylinders 36A-36D in the compressor 10 in the illustrated embodiment. Referring to FIGS. 2A and 3A, the charging ports 122A-122D establish fluid communication between the cylinders 36A-36D and the component chamber 114. In the illustrated embodiment, a single charging port 122 is associated with each one of the cylinders 36. Thus, the first cylinder 36A has a first charging port 122A, the second cylinder 36B has a second charging port 122B, the third cylinder 36C has a third charging port 122C, and the fourth cylinder 36D has a fourth charging port 122D.

A plurality of components are located in the component chamber 114 of the cylinder heads 110A, 110B. The components direct fluid flow between the inlet 118 of the first head 110A, the cylinders 36A-36D and the outlet 120 of the second head 110B. The components include a plurality of check valves 130A-130F for controlling flow of air into and out of the various cylinders 36A-36D, and a plurality of components or structures for positioning the check valves in the chamber 114 and inhibiting gas flow around the check valves (i.e. leakage around the check valves). In one exemplary embodiment, the components for positioning the check valves are spacers and are configured to direct air to flow between the check valves. The check valves may also be spaced apart in a variety of ways, other than using spacers. For example, one or more of the check valves may thread into the component chamber 114, the component chamber may include a stop surface, etc. Any manner of positioning the check valves may be used. In the drawings, arrangements for setting the position of the check valves with respect to the inlets 118 and outlets 120 of the cylinder heads 110A, 110B are not shown. However, it is understood that spacers or another positioning arrangement would be used to position the illustrated check valves and spacers as shown. For example, U.S. Patent Application Publication, Pub. No. 2007/0065301 shows that inlet and outlet connectors 180, 196 may engage spacers that fix the position of the valves. The components located in the component chamber may also include a plurality of seals that prevent leakage around the check valves.

As shown in FIGS. 9 and 10, the check valves 130A-130F that are in the cylinder heads 110A, 110B are preferably identical to each other. Other types of check valves than that shown can be used. Referring to FIGS. 9 and 10, each illustrated check valve 130A-130F includes a valve body 132 having a generally cylindrical configuration with a central chamber 134. An end wall 136 is located at the upstream end of the valve body 132. The end wall 136 has a central opening 138. The downstream end of the valve body 132 is open. The check valve 130A-130F each include a movable valve element in the form of a ball 146. The dimensions of the ball 146 are selected so that when the ball is in engagement with the end wall 136 of the valve body 132, the ball closes the opening 138. When the ball 146 is away from the end wall 136, fluid flow is enabled through the check valve. A spring biases the ball into engagement with the end wall 136 to close the valve. Further details of acceptable check valves are described in U.S. Patent Application Publication No. 2007/0065301.

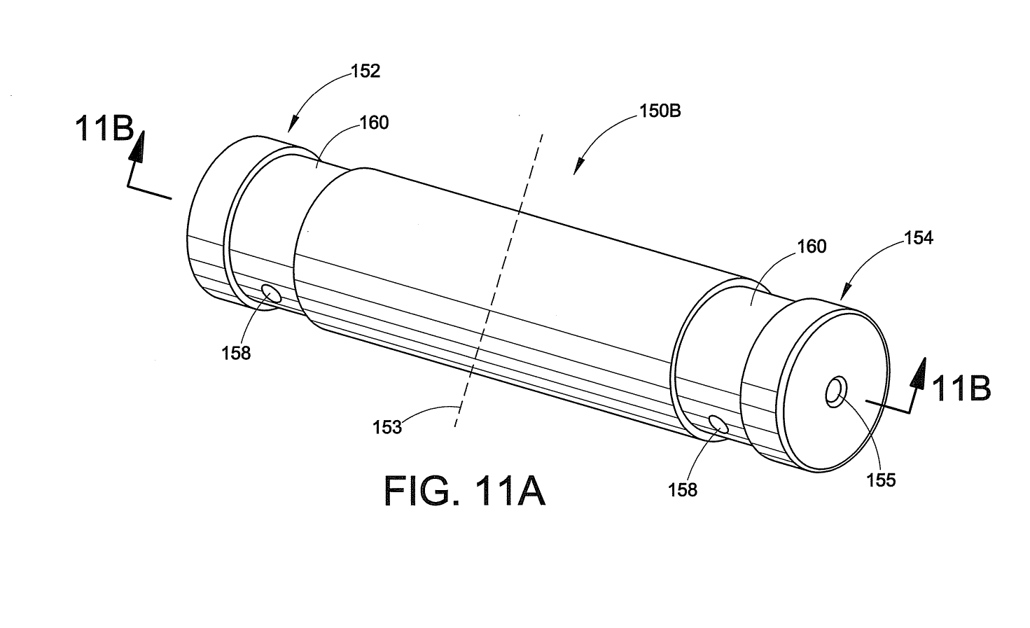

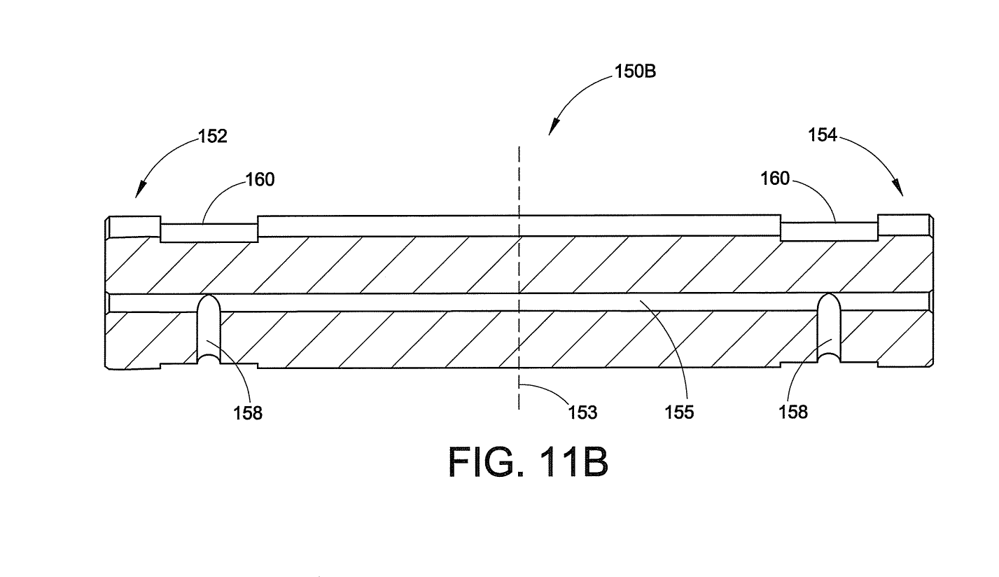

Spacers 150A-150D are positioned in the chamber 114 and space the check valves 130A-130F apart. FIGS. 11A and 11B illustrate the spacers 150B-150D. The spacers 150B-150D are preferably identical to each other. Each spacer 150B-150D is a cylindrical block of metal that has an outside diameter substantially equal in size to the inside diameter of the component chamber 114 in the cylinder heads 110A, 110B. The spacers 150B-150D has an upstream end portion 152 and a downstream end portion 154. However, in the illustrated embodiment, the end portions 152, 154 are identical, since the spacer is symmetrical about a midplane 153.

In the embodiment illustrated in FIGS. 11A and 11B, the spacer 150 has a small diameter central opening 155 that extends for the length of the spacer between the upstream end portion 152 and the downstream end portion 154. The symmetric end portions 152, 154 both include passages 158 that extend radially outward from the central opening 155 and an external groove 160 in fluid communication with the passage 158. As a result, fluid communication is established between the central opening 155 of the spacer 150, and the external groove 160.

Referring to FIG. 9, the spacer 150A is shorter than the spacers 150B-150D. The spacer 150A is a cylindrical block of metal that has an outside diameter substantially equal in size to the inside diameter of the component chamber 114 in the cylinder head 110. The spacer 150A has symmetrical upstream and downstream end portions 164, 166.

A small diameter central opening 170 extends for the length of the short spacer between the upstream end portion 164 and the downstream end portion 166. The spacer 150A also has an internal passage 172 that extends radially outward from the central passage 170 and terminates in a groove 174 on the outer surface of the spacer 150A. As a result, fluid communication is established between the upstream and downstream end portions 164 and 166 of the spacer 150A, and the external groove 174.

As shown in FIGS. 9 and 10, an inlet connector 180 is secured in the upstream end of each of the cylinder heads 110A, 110B. The inlet connector has a fluid inlet passage 182 that communicates with the component chamber. An outlet connector 196 is secured in the downstream end of each of the cylinder heads 110A, 110B. The outlet connector 196 has a fluid outlet passage 198 that communicates with the component chamber 114. The components are positioned in the component chamber 114 in the cylinder heads 110A, 110B.

An inlet check valve 130E is positioned in the component chamber 114 in the first cylinder head 110A. The inlet opening 138 of the inlet check valve 130E is in communication with the inlet 118 of compressor 10. In an exemplary embodiment, a seal may be provided between the check valve and the component chamber 114.

The spacer 150A is positioned in the component chamber 114 in the cylinder head 110 such that an upstream end of the spacer 154A engages the downstream end of the inlet check valve 130E. The external groove 174 on the spacer 162 aligns with the first charging port 122A in the cylinder head 110A. As a result, fluid communication can be established between the component chamber 114 and the first cylinder 36A. (See FIG. 2A).

Referring to FIG. 9, a second check valve, or first cylinder check valve, 130A is positioned in the component chamber 114 in the cylinder head 110A. The upstream end of the second check valve 130A engages the downstream end of the spacer 150A. The inlet opening 138 of the second check valve 130A aligns with the central passage 170 in the spacer 150B. An optional seal is provided between the spacer 150A and the second check valve 130A.

Referring to FIG. 9, a spacer 150B is positioned in the component chamber 114 in the cylinder head 110A. The upstream end of the spacer 150B engages the downstream end of the check valve 130A. The central opening 155 of the spacer 150B aligns with the outlet of the check valve 130A. The external groove 160 at the downstream end of the second spacer 150B aligns with the second charging port 122B in the cylinder head 110A. As a result, fluid communication is established between the component chamber 114 and the second cylinder 36B (See FIG. 2A).

Referring to FIG. 9, a third check valve, or second cylinder check valve, 130B is positioned in the component chamber 114 in the cylinder head 110A. The upstream end of the check valve 130B engages the downstream end of the spacer 150B. The opening 138 of the check valve 130B aligns with the central passage 155 in the spacer 150B. An optional seal is formed between the spacer 150B and the check valve 130B.

Referring to FIG. 10, an optional fourth check valve, or second head inlet check valve 130C is positioned in the component chamber 114 in the second cylinder head 110B. The inlet opening 138 of the inlet check valve 130C is in communication with the inlet 118 of second head 110B. In an exemplary embodiment, a seal may be provided between the check valve and the component chamber 114.

A spacer 150C is positioned in the component chamber 114 in the cylinder head 110B. The upstream end of the spacer 150C engages the downstream end of the check valve 130C. The central opening 155 of the spacer 150C aligns with the central opening of the check valve 130C. The external groove 160 of the spacer 150C aligns with the charging port 122C in the cylinder head 110B. As a result, fluid communication can be established between the component chamber 114 and the third cylinder 36C (See FIG. 3A).

A fifth check valve, or third cylinder check valve, 130D is positioned in the component chamber 114 in the cylinder head 110B. The upstream end of the check valve 130D engages the downstream end of the spacer 150C. The opening 138 of the check valve 130D aligns with the passage 155 in the spacer 150C. A seal may be provided between spacer 150C and the check valve 130D.

A spacer 150D is positioned in the component chamber 114 in the cylinder head 110B. The upstream end of the spacer 150D engages the downstream end of the third cylinder check valve 130D. The central opening 156 of the spacer 150D aligns with the central chamber of the check valve 130D. The external groove 160 at the downstream end of the fourth spacer 150D aligns with the fourth charging port 122D in the cylinder head 110. As a result, fluid communication can be established between the component chamber 114 and the fourth cylinder 36D.

A sixth check valve, or fourth cylinder check valve 130F is positioned in the component chamber 114 in the cylinder head 110B. The upstream end of the fourth cylinder check valve 130F engages the downstream end of the spacer 150D. The opening 138 of the check valve aligns with the central passage 155 in the spacer 150D. An optional seal is provided between the spacer 150D and the check valve 130D.

An outlet connector 196 is fixed to the downstream end of the cylinder head 110B. The outlet connector 196 has a fluid outlet passage 198 that is in fluid communication with the component chamber 114 of the cylinder head 110B. In the illustrated embodiments, all the check valves 130A-F of the compressor 10 are located in the cylinder heads 110A, 110B.

Referring once again to FIGS. 2A and 3A, when the compressor 10 is operating, air is admitted to the compressor through the inlet connector 180 of the first head 110A. The air flows through the inlet connector 180 of the first head 110A and to the inlet check valve 130E.

When the compressor 10 is at the portion of its cycle in which the first cylinder 36A is on the intake phase, the pressure in the first cylinder is lower than the intake pressure. As a result, intake gas flows through the inlet check valve 130E and into the spacer 150A.

The gas flows from the central passage 170 (See FIG. 9) of the spacer 150A, radially outward through the passage 172, into the external groove 174 on the spacer. The air then flows through the first charging port 122A and into the first cylinder 36A (See FIG. 2A).

Referring to FIGS. 2A and 9, during this time, the gas flowing through the inlet check valve 130E does not flow through the second check valve 130A, even though the spacer 150A is open for free flow to the second check valve. This is because the pressure downstream of the second check valve 130A, i.e., the pressure in the second cylinder 36B, is higher than the intake pressure. Therefore, the second check valve 130A stays closed and the intake air flows into the first cylinder 36A.

When the first piston 40A thereafter is compressing the air in the first cylinder 36A, the pressure in the first cylinder becomes higher than the intake pressure. As a result, intake air can not flow upstream through the inlet check valve 130E into the spacer 150A. Therefore, all the air flowing out of the first cylinder is directed through the first charging port 122A, the spacer 150A, and through the second check valve 130A.

Referring to FIGS. 2A and 9, the second check valve 130A is forced open to allow air to flow out of the first cylinder 36A into the second spacer 150B. The air flows through the second spacer 150B to the radially extending passages 158 (See FIGS. 11A and 11B) and the external groove 160 in the downstream end 154 of the second spacer 150B. The air then flows from the groove 160 into the second charging port 122B.

The timing of the first and second cylinders 36A and 36B is selected so that when the first cylinder 36A is on its exhaust phase, the second cylinder 36B is on its intake phase. This is achieved by the 180 degree offset β between the first and second eccentric bodies 84A, 84B. The air that is compressed in the first cylinder 36A and forced into the second spacer 150B is able to flow into the second cylinder 36B, to be further compressed, because the second cylinder is smaller in diameter than the first cylinder but has the same stroke in the illustrated exemplary embodiment.

During the time the second cylinder 36A is being charged by the first cylinder 36B, the air flowing through the second spacer 150B does not flow through the third check valve 130B, even through the second spacer is open to the third check valve. This is because the pressure downstream of the third check valve 130B, (i.e., the pressure in the third cylinder 36C), is higher than the pressure at the third check valve. Therefore, the third check valve 130B stays closed and the air flows into the second cylinder 36B.

Referring to FIGS. 3A and 10, in a similar manner, the air that is compressed in the second cylinder 36B flows through the conduit 119 into the third cylinder 36C, there to be further compressed. The air that is compressed in the third cylinder 36C flows into the fourth cylinder 36D, there to be further compressed. The air that is compressed in the fourth cylinder 36D flows out of the compressor 10 through the outlet connector 194.

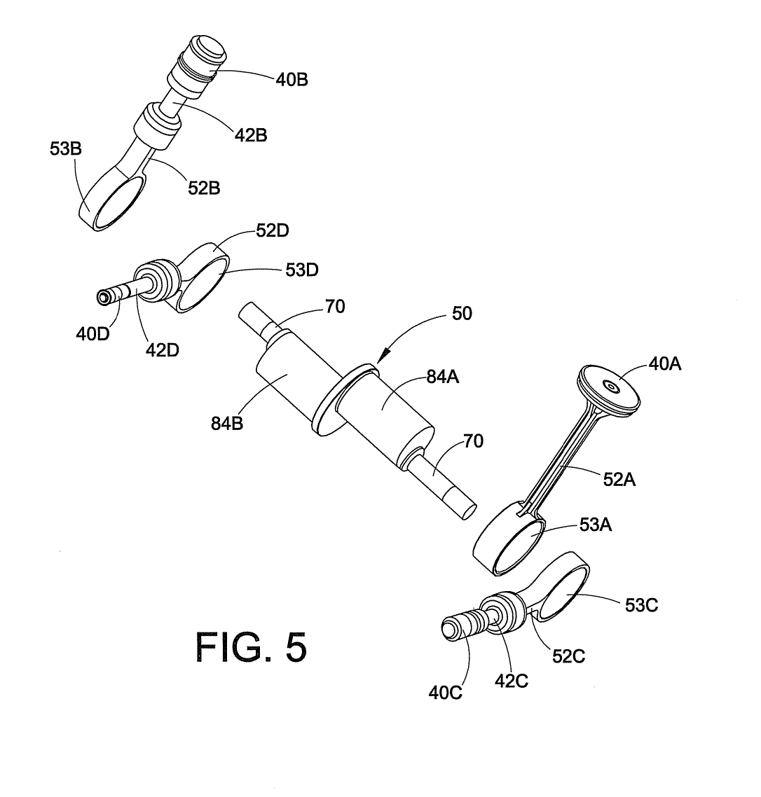

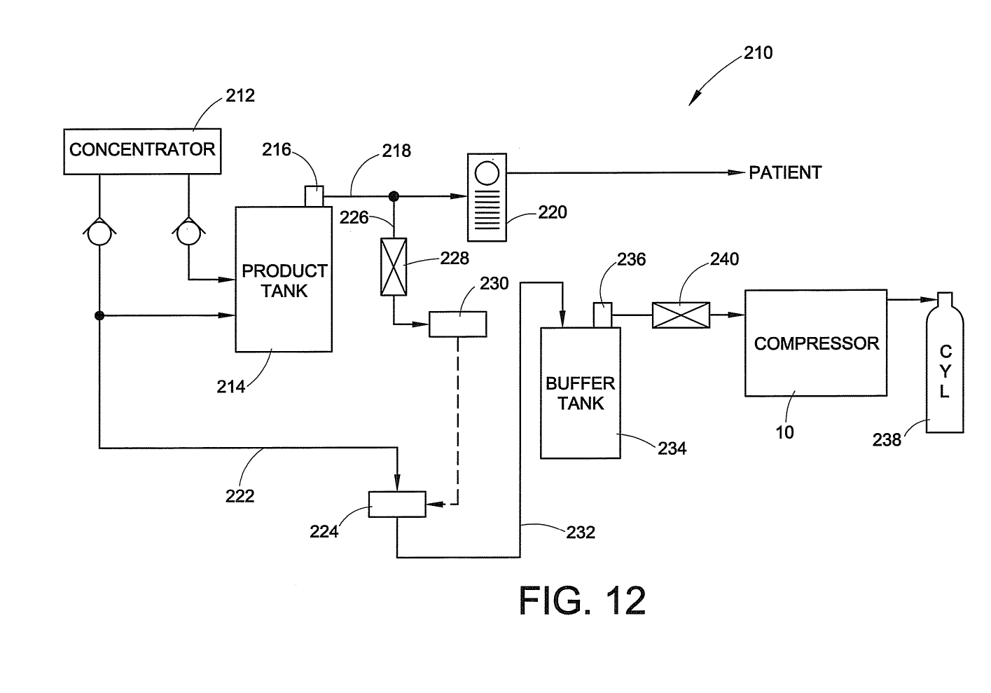

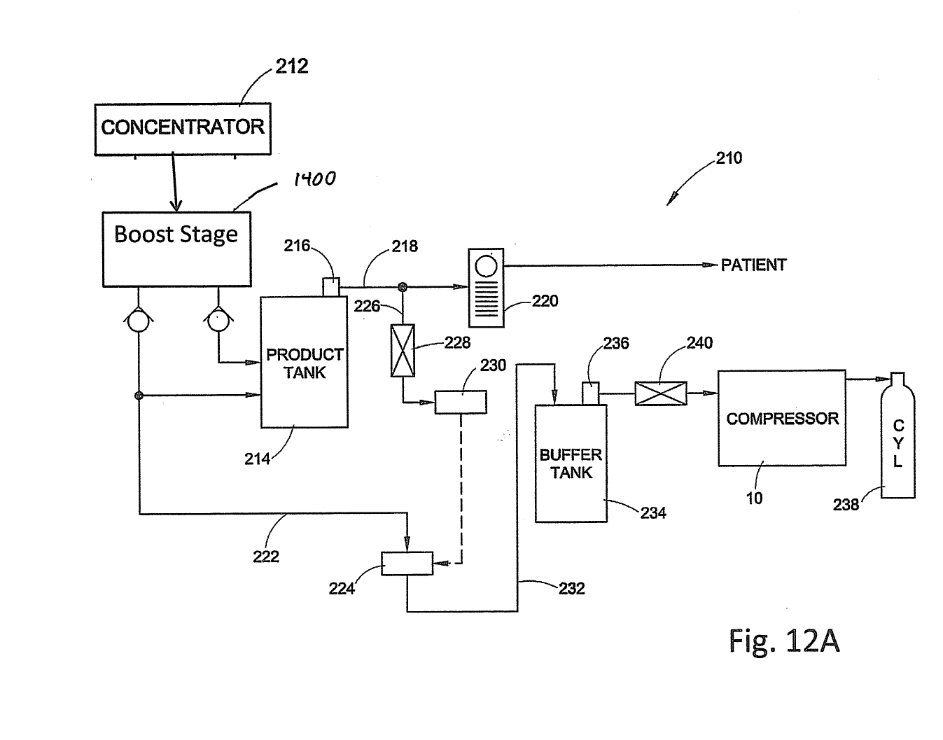

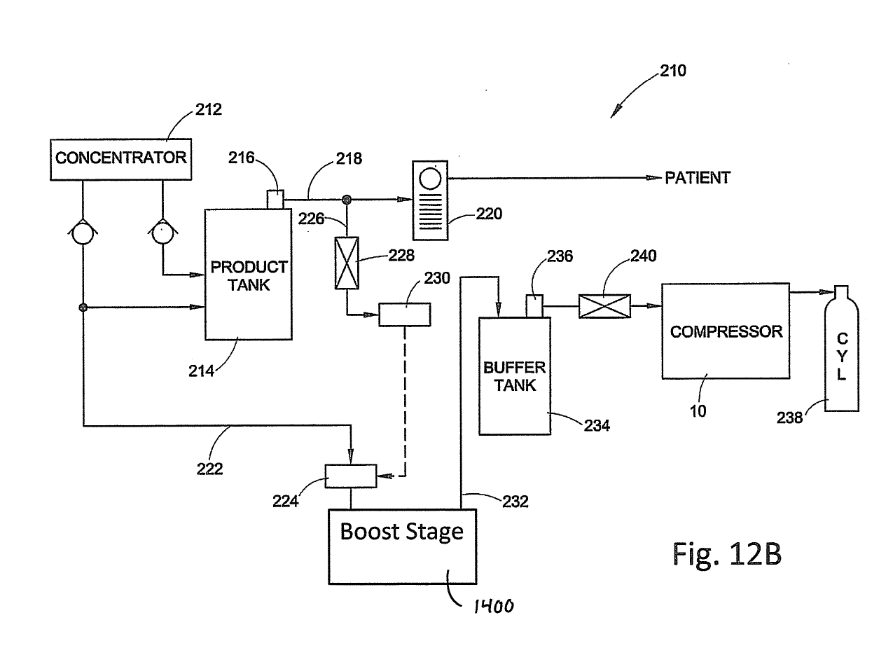

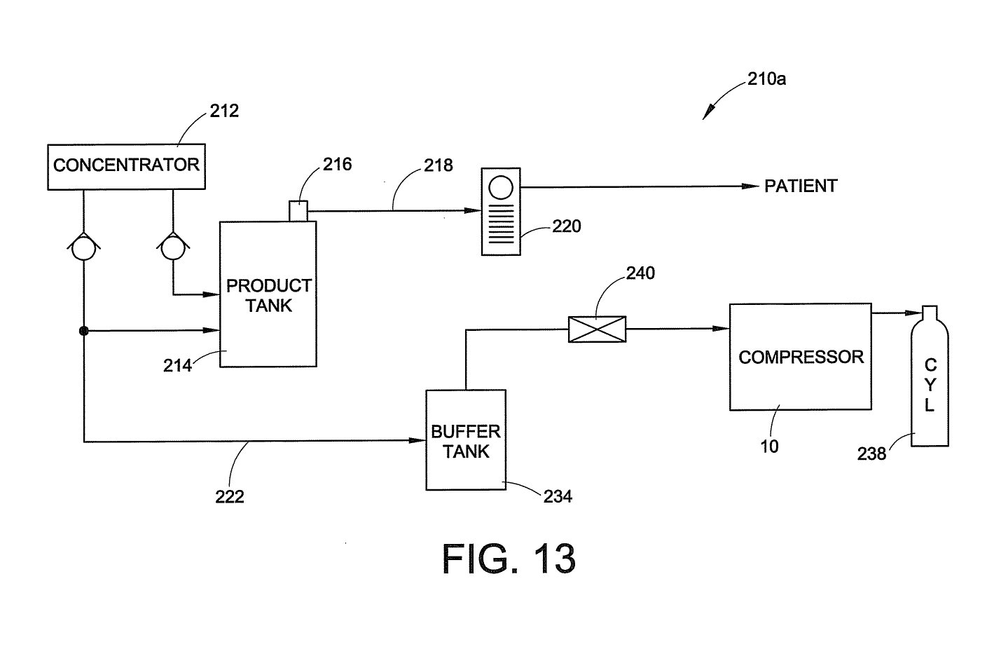

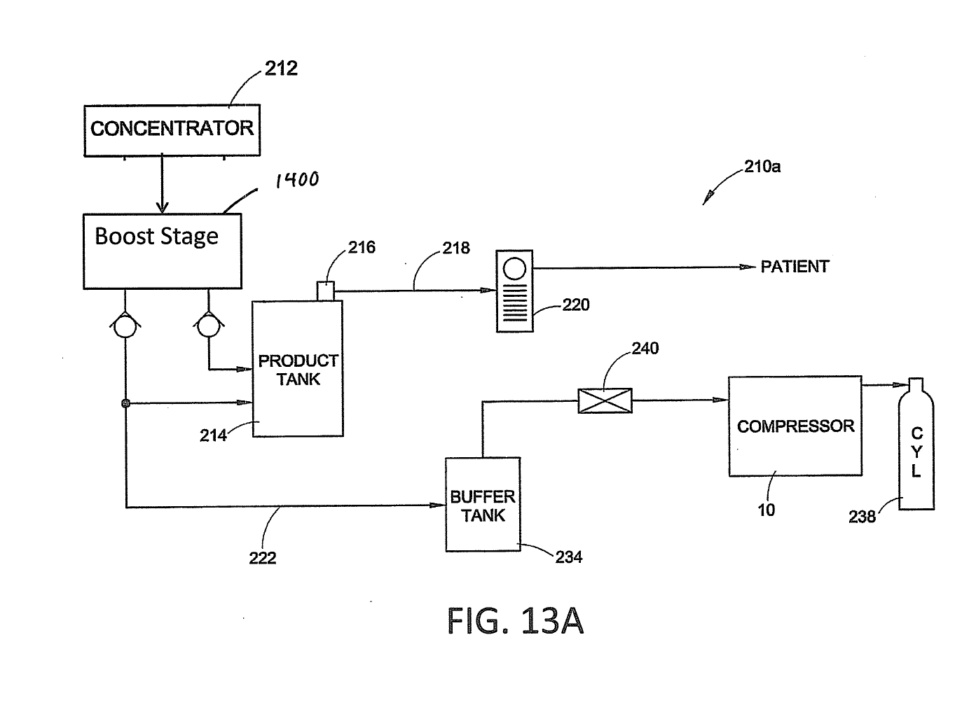

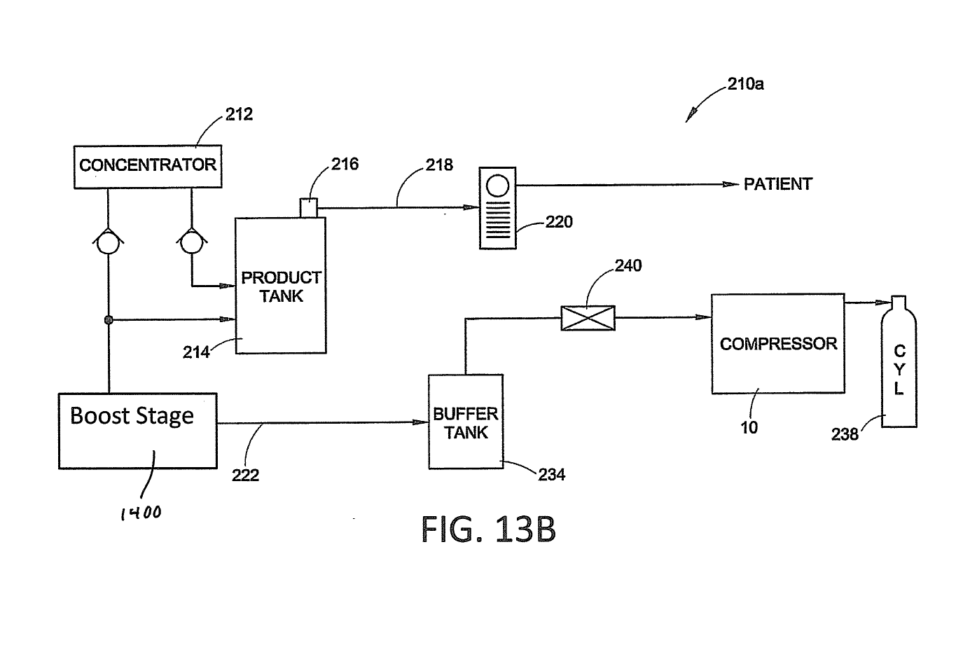

Referring to FIG. 12, a system 210 includes a concentrator 212 that is operable to provide oxygen-enriched gas, for example, from an ambient air input. The oxygen-enriched gas is fed to a product tank 214. A regulator 216 emits oxygen-enriched gas from the product tank 214 into a flow line 218 and feeds the same to a flow meter 220 which subsequently emits the oxygen-enriched gas to the patient at a predetermined flow rate, for example a flow rate of from 0.1 to 6 liters per minute. Optionally, the flow meter 220 can be closed so that all the oxygen-enriched gas is directed to the compressor 10. The compressor may take a wide variety of forms and may include any combination or subcombination of the features of the compressors described with respect to FIGS. 1-11. Further, any combination or subcombination of the features of the compressors described with respect to FIGS. 1-11 can be used in a wide variety of different applications, including but not limited to the systems illustrated by FIGS. 12 and 13.

Gas not directed to the patient is carried via line 222 to two-way valve 224. A very small portion of the gas in the flow line 220 is directed through line 226 and restrictor 228 into an oxygen sensor 230 which detects whether or not the concentration of the oxygen is of a predetermined value, for example, at least 84 percent as directed to the patient and at least 93±3% as directed to the compressor.

When the oxygen sensor 230 detects a concentration at or above the predetermined level, the two-way valve 224 is kept open to permit the oxygen-enriched gas to flow through the valve 224 and line 232 into a buffer tank 234 wherein the pressure is essentially the same as the pressure in the product tank 214. However, should the oxygen sensor 230 not detect a suitable oxygen concentration, two-way valve 224 is closed so that the oxygen concentrator 212 can build up a sufficient oxygen concentration. This arrangement prioritizes the flow of oxygen-enriched gas so that the patient is assured of receiving a gas having a sufficient oxygen concentration therein.

Buffer tank 234 can have a regulator 236 thereon generally set at 12 psi to admit the oxygen-enriched gas to the compressor 10 when needed. The output of the compressor 10 is used to fill a cylinder or portable tank 238 for ambulatory use by the patient. Alternatively, the pressure regulator 236 can be set at anywhere from about 13 to about 21 psi. A restrictor 240 controls the flow rate of gas from the buffer tank 234 to the compressor 10. Should the operation of the compressor 10 cause the pressure in the buffer tank 234 to drop below a predetermined value, a pressure sensor (not shown) automatically cuts off the flow of gas at a pressure above the pressure of the gas being fed to the patient. This prioritization assures that the patient receives priority with regard to oxygen-enriched gas.

FIG. 13 shows a system 210a that is somewhat different from the system 210 of FIG. 12. In the system 210a, the compressor 10 includes its own oxygen sensor and control circuitry, so that the elements 224-232 are not present as they are in the system shown in FIG. 12. In addition, the regulator 236 is not present on the buffer tank. A flow restrictor may be provided between the concentrator and the buffer tank. (It should be noted that the buffer tank 234 is optional in all systems, and that the compressor could be fed directly from the product tank).

In one exemplary embodiment, a boost stage 1400 is disposed in a fluid circuit between the oxygen concentrator 212 and the compressor 10 (See FIGS. 12A, 12B, 13A, and 13B). The boost stage 1400 pre-charges or pre-compresses the first stage of the compressor 10. Different models of concentrators 212 provide concentrated oxygen at different pressures. These differences in pressure can cause a variance in the amount of time required to fill a portable tank or cylinder 238. In addition, variables, such as patient regulator pressure settings and altitude can also cause variances in the amount of time required to fill a portable tank or cylinder 238. The boost stage 1400 provides the concentrated oxygen from the concentrator 212 to the compressor 10 at an elevated, constant pressure. Providing the concentrated oxygen to the compressor 10 at a constant pressure reduces the variance in the amount of time needed to fill a portable tank or cylinder 238. In addition, providing the concentrated oxygen to the compressor 10 at a pressure that is higher than is typically available from the oxygen concentrator may allow the compressor to operate at a higher efficiency. For example, concentrated oxygen from the oxygen concentrator is typically provided to the compressor 10 at about 5 psi. In one exemplary embodiment, the boost stage 1400 provides oxygen to the to the compressor at 10-20 psi, such as at about 15 psi.

The boost stage 1400 may take a wide variety of different forms and may be used with a wide variety of different compressors. The boost stage 1400 may be used with the compressor 10, or any other compressor or any other pressure increasing device. The boost stage 1400 may take a wide variety of different forms. The boost stage 1400 may be any arrangement that increases the pressure of the concentrated oxygen from the concentrator 212 and controls a maximum pressure of the concentrated oxygen provided to the compressor 10.

In the exemplary embodiment illustrated by FIG. 14, the boost stage 1400 includes a pressure increasing device 1402 and a pressure limiting device 1404. The boost stage 1400 may also include an optional check valve 1406 that prevents concentrated oxygen from flowing back toward the concentrator 212 and an optional accumulator or buffer tank 1408. In the boost stage, concentrated oxygen from the concentrator 212 is provided through a line 1409 to the pressure increasing device 1402 as indicated by arrow 1410. The pressure increasing device 1402 increases the pressure of the concentrated oxygen. The increased pressure concentrated oxygen flows in a line 1414 from the pressure increasing device 1402 to the inlet to the compressor 10 as indicated by arrow 1415 (and through the accumulator 1408, if included).

The illustrated pressure limiting device 1404 is disposed between the flow lines 1409 and 1414. However, the pressure limiting device 1404 may take a wide variety of different forms and may arranged in the boost stage in a wide variety of different ways. In the example illustrated by FIG. 14, the pressure limiting device 1404 opens a line 1416 between the line 1409 and the line 1404 when the pressure in the line 1414 reaches a predetermined pressure set point. When the pressure in the line 1414 is less than the predetermined pressure set point, the line 1416 is closed. When the line 1416 opens, the increased pressure concentrated oxygen flows as indicated by arrow 1418 from the line 1414 back into the line 1409. As such, the pressure limiting device 1404 inhibits the pressure of the concentrated oxygen provided to the compressor 10 from exceeding the predetermined pressure set point. When the output of the pressure increasing device 1402 is at least as high as the predetermined pressure set point, the boost stage 1400 substantially regulates the pressure provided to the compressor 10 at the predetermined pressure set point.

The pressure increasing device 1402 can take a wide variety of different forms. The pressure increasing device 1402 may be any device capable of increasing the pressure of the concentrated oxygen from the concentrator. Examples of pressure increasing devices include, but are not limited to, compressors, pressure intensifiers, pumps, blowers, fans, and the like. Referring to FIG. 15, in one exemplary embodiment, the pressure increasing device 1402 is single stage compressor 1500. The single stage compressor 1500 can take a wide variety of different forms. In the example illustrated by FIG. 15, the compressor 1500 includes a cylinder 1502 and a piston 1504. The piston 1504 reciprocates as indicated by arrow 1506 in the cylinder 1502 to draw in concentrated oxygen from line 1409, compress the concentrated oxygen, and provide compressed concentrated oxygen to the line 1414.

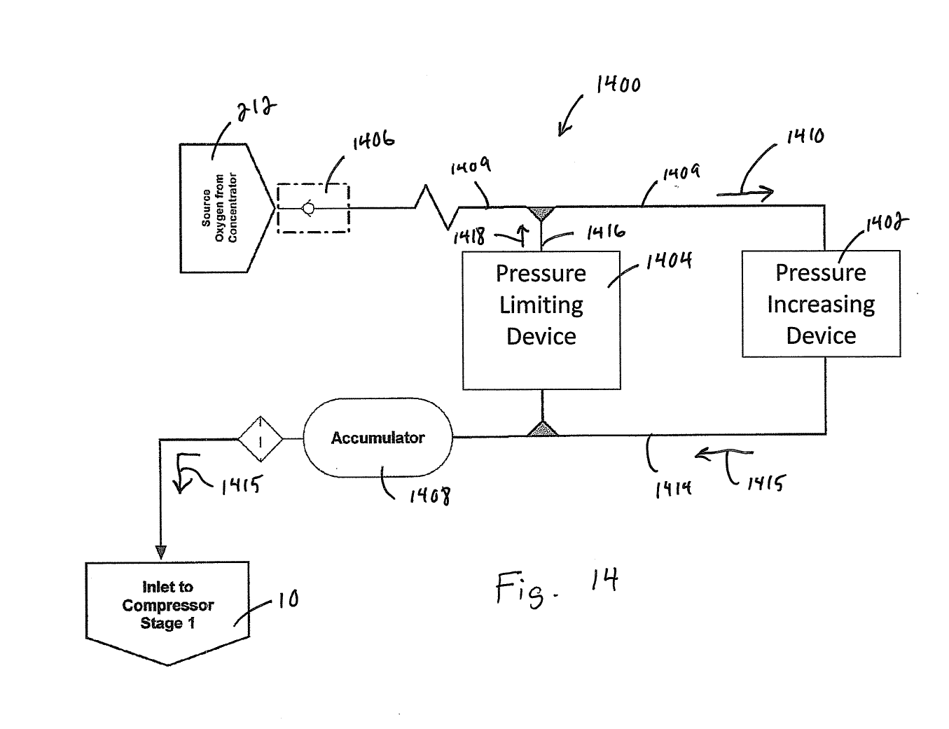

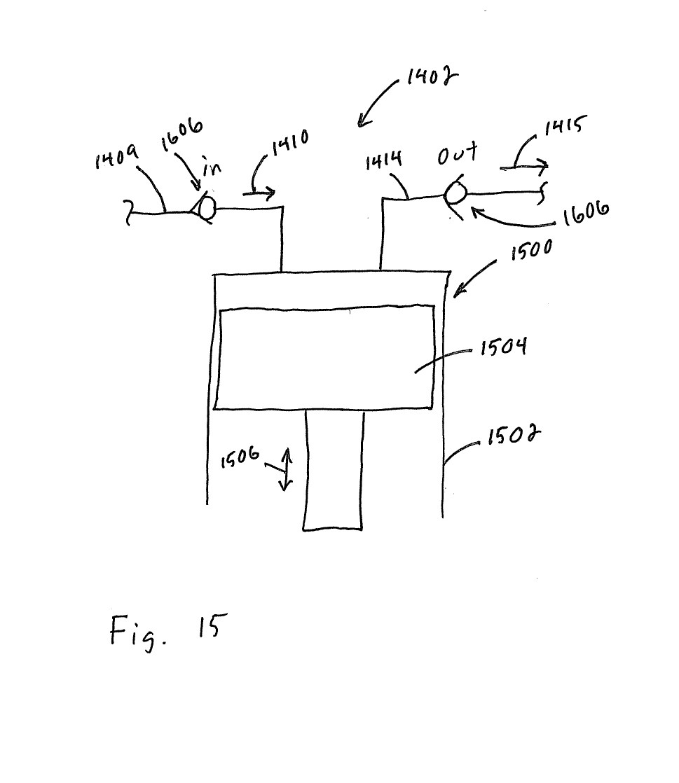

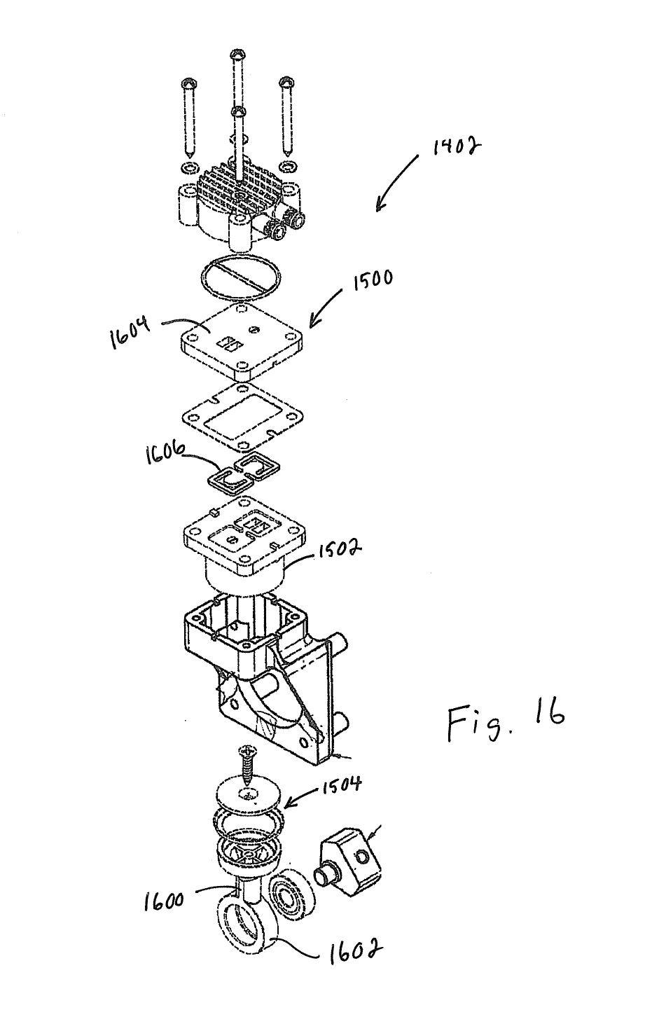

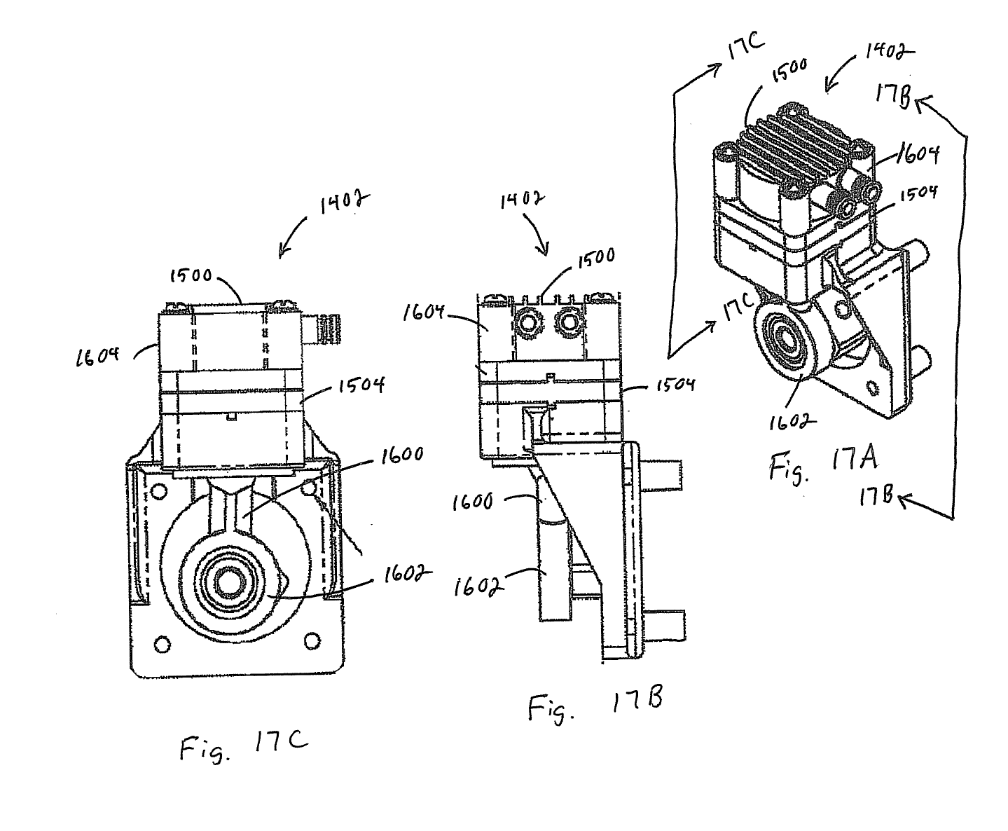

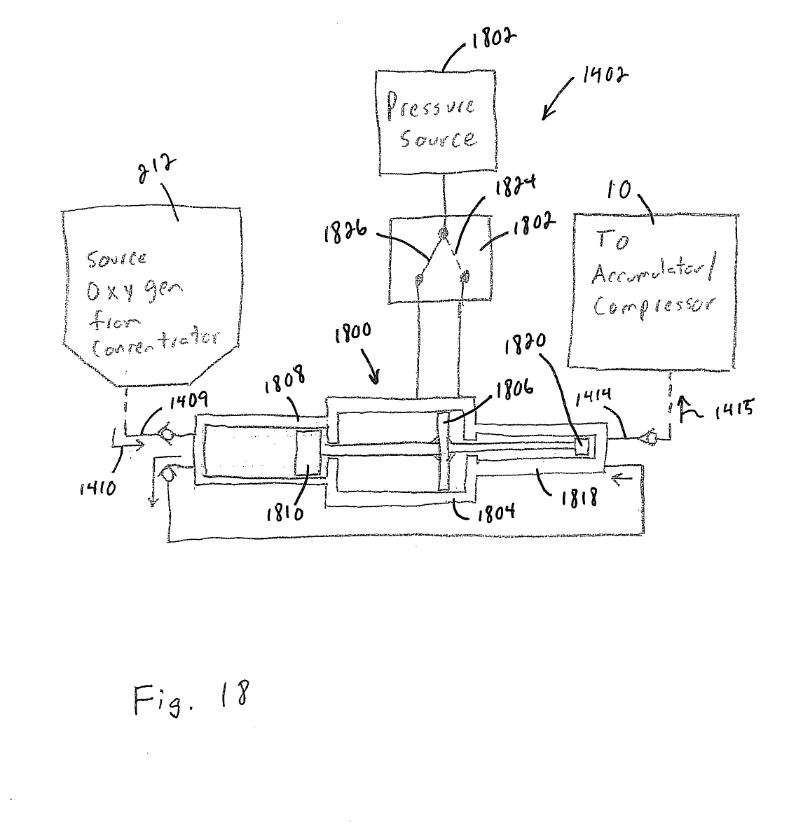

FIG. 16 is an exploded perspective view of an example of one of the many different single stage compressors 1500 that can be used. FIGS. 17A-17C illustrate the single stage compressor 1500 shown in FIG. 16 in an assembled condition. The single stage compressor 1500 includes a cylinder 1502, a piston 1504, and a piston rod 1600 with a ring 1602. The piston rod 1600 with ring 1602 reciprocates the piston 1504 in the cylinder when an eccentric rotational movement is imparted onto the ring 1602. A head 1604 includes a check valve arrangement that 1606 prevents concentrated oxygen from flowing from line 1414 back into the cylinder 1502 and back from the cylinder 1502 into the line 1409.

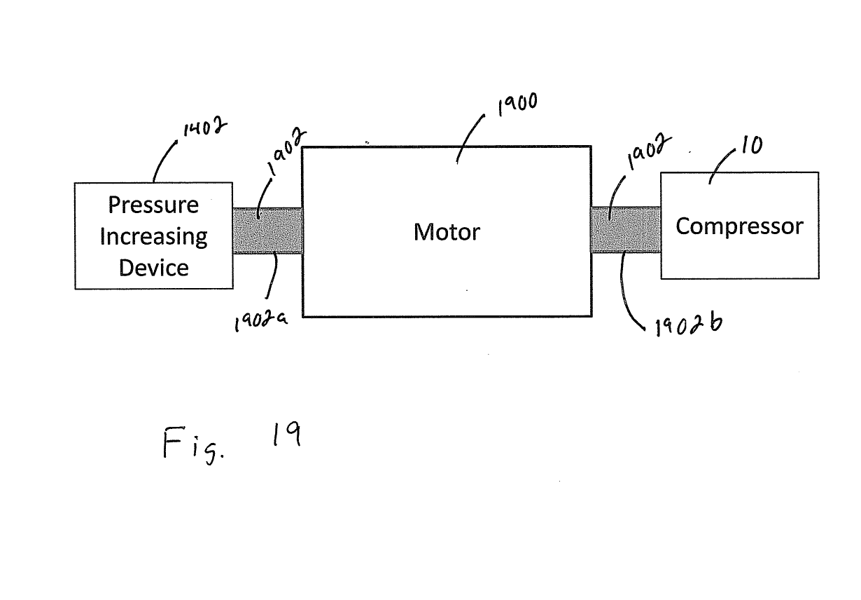

FIG. 18 illustrates another example of a pressure increasing device 1402. In the example illustrated by FIG. 18, the pressure increasing device 1402 is a pressure intensifier 1800. The pressure intensifier may take a wide variety of different forms. In the example illustrated by FIG. 18, the pressure intensifier 1800 is powered by a source of pressure 1802, such as the source of compressed air that feeds the concentrator 212, the concentrated oxygen from the concentrator 212, or another source of compressed fluid. The illustrated pressure intensifier 1800 is a two-stage pressure intensifier. However, the pressure intensifier can be a single stage pressure intensifier or the pressure intensifier may have more than two stages. The illustrated pressure intensifier 1800 includes a switching valve 1802, a drive cylinder 1804, a drive piston 1806, a first stage cylinder 1808, first stage piston 1810, a second stage cylinder 1818, and a second stage piston 1820. The concentrator 212 provides concentrated oxygen through the line 1409 to the first stage cylinder 1808 as indicated by arrow 1410. When the switching valve 1802 is in the position 1824, the first stage piston 1810 compresses the concentrated oxygen in the first stage cylinder 1810 and provides the concentrated oxygen to the second stage cylinder 1818. When the switching valve 1802 is in the position 1826, the second stage piston 1820 compresses the concentrated oxygen in the second stage cylinder 1820 and provides the compressed concentrated oxygen to the compressor 10 through the line 1414.

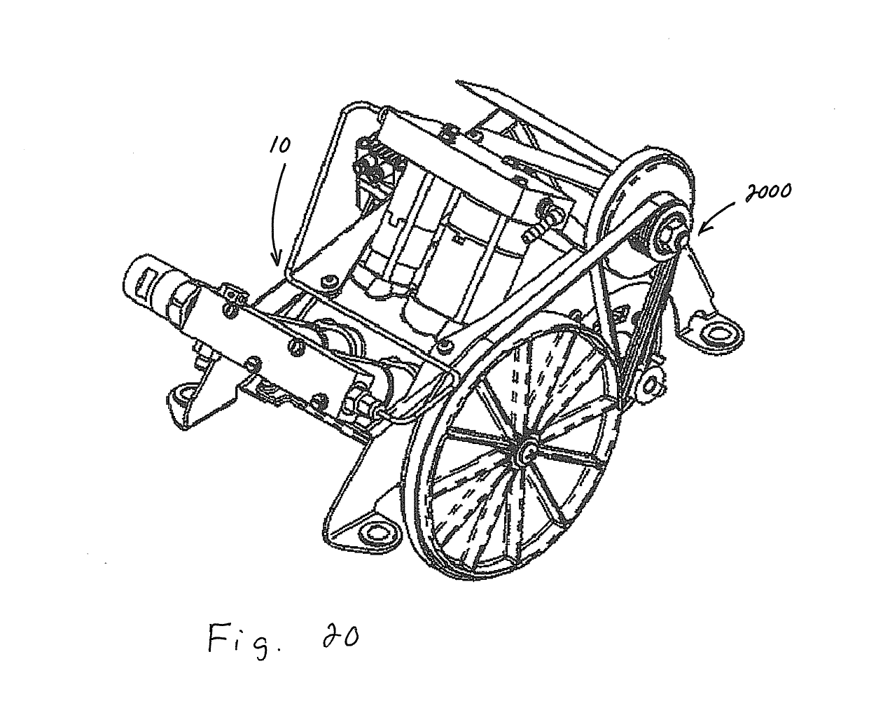

The pressure increasing device 1402 can be powered or driven in a wide variety of different ways. For example, pressure increasing device 1402 can be driven by the same motor 1900 that drives the compressor 10 or the pressure increasing device 1402 can be driven by a device that is separate or independent from the motor 1900 that drives the compressor 10. In the example illustrated by FIG. 19, a drive shaft 1902 of the motor 1900 rotates to drive both the compressor 10 and the pressure increasing device 1402. In the FIG. 19 example, portions 1902a, 1902b on opposite sides of the motor 1900 drive the pressure increasing device 1402 and the compressor 10 respectively. In another embodiment, a portion of a drive shaft on one side of the motor drives both the pressure increasing device 1402 and the compressor 10 and a second shaft portion, on the opposite side of the motor, may not be included. Referring to FIG. 15, in one exemplary embodiment, the pressure increasing device 1402 is single stage compressor 1500.

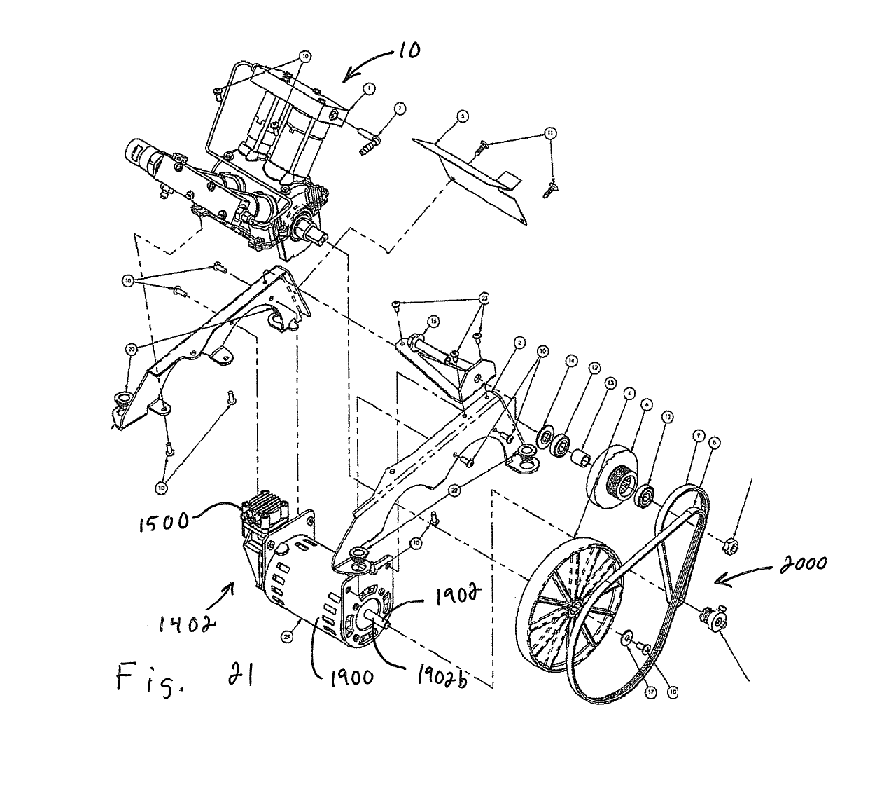

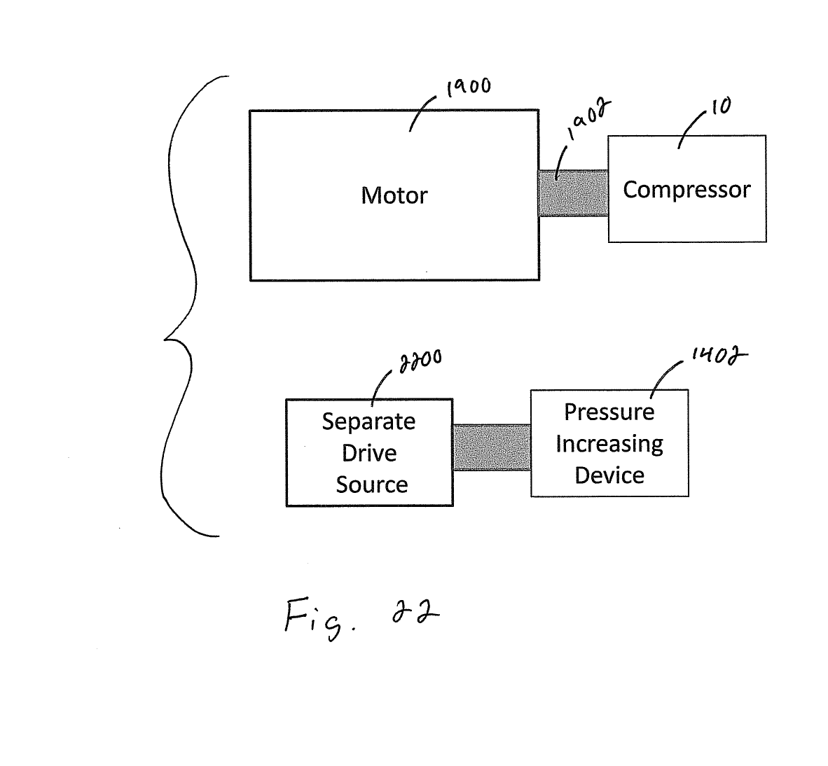

FIGS. 19 and 20 illustrate an example where the same motor 1900 drives both the pressure increasing device 1402 and the compressor 10. However, a wide variety of other arrangements can be used to drive the pressure increasing device 1402 and the compressor 10 with the same motor 1900. In the example illustrated by FIGS. 20 and 21, the compressor 10 has the four cylinder, V configuration described above and the pressure increasing device 1402 is a single stage compressor 1500. An output shaft, not shown, on one side of the motor 1900 drives the single stage compressor 1500. An output shaft 1902b on the other side of the motor 1900 drives the compressor 10 through a belt and pulley arrangement 2000.

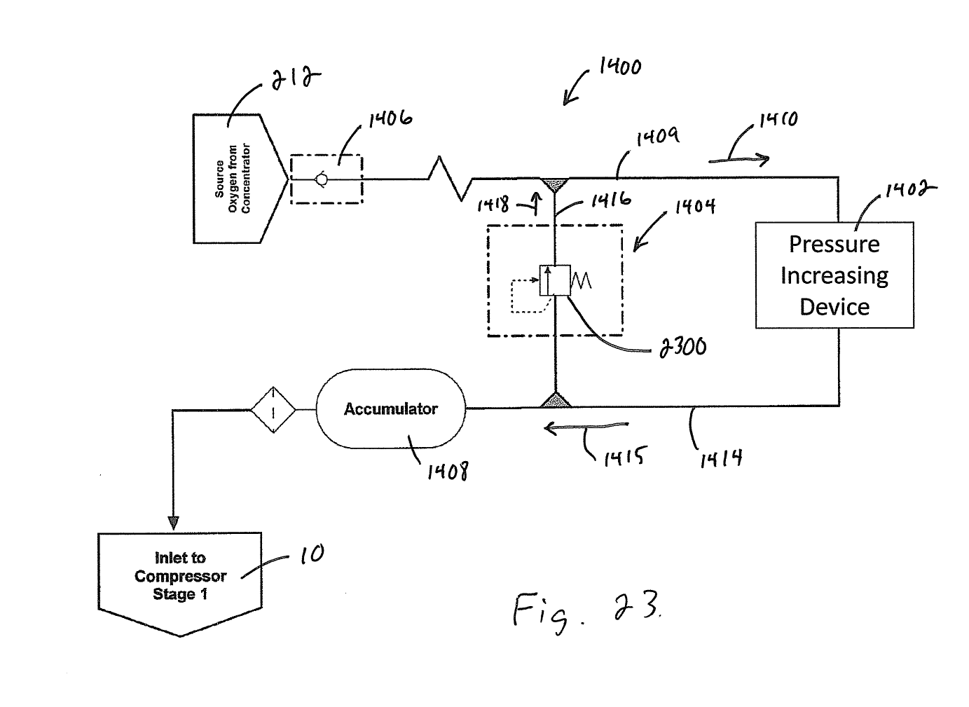

FIG. 22 illustrates an example where a separate drive source 2200, that is independent from the motor 1900 that drives the compressor 10, drives the pressure increasing device 1402. The separate drive source 2200 can take a wide variety of different forms. Examples of separate drive sources 2200 include, but are not limited to motors, sources of fluid pressure, electromagnetic actuators, and the like. The pressure source 1802 and pressure intensifier 1800 illustrated by FIG. 18 are one example of a separate drive source that drives the pressure increasing device 1402. In the example illustrated by FIG. 22, the drive shaft 1902 of the motor 1900 rotates to drive the compressor 10. The separate drive source 2200 drives the pressure increasing device 1402.

The pressure limiting device 1404 can take a wide variety of different forms. The pressure limiting device 1404 may be any device or arrangement capable of limiting the pressure applied to the compressor 10 or any device or arrangement capable of limiting the differential pressure between the line 1414 and the line 1409. Examples of pressure limiting devices 1404 include, but are not limited to, regulators, check valves, valve and pressure sensor arrangements, and pressure sensor arrangements that control operation of the pressure increasing device.

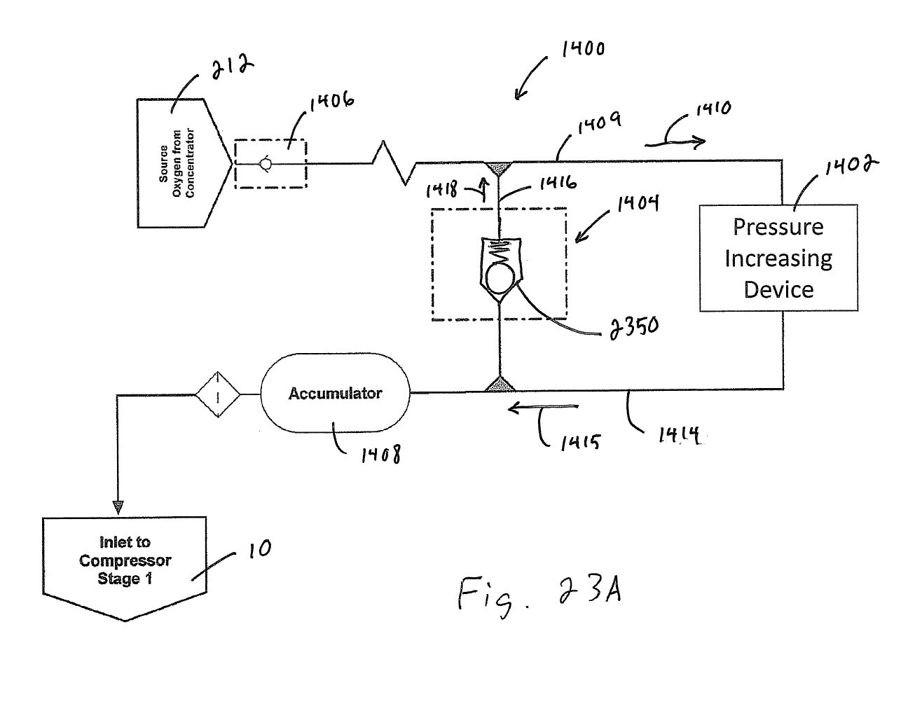

Referring to FIG. 23, in one exemplary embodiment, the pressure limiting device 1404 is a regulator 2300, such as a mechanical regulator or an electro-mechanical regulator. When the pressure in the line 1414 is below the pressure set point, the regulator 2300 closes the line 1416. When the pressure in the line 1414 is greater than the pressure set point, the regulator 2300 opens the line 1416.

FIG. 23A illustrates an exemplary embodiment where the pressure limiting device 1404 limits the differential pressure between the line 1414 and the line 1409, rather than setting the pressure in the line 1414. A wide variety of different devices can be used to limit the differential pressure between the line 1414 and the line 1409. In the example illustrated by FIG. 23A, the pressure limiting device is a check valve 2350. The check valve 2350 is constructed to open the line 1416 when the pressure in the line 1414 minus the pressure in the line 1409 is greater than the pressure differential set point. When the pressure in the line 1414 minus the pressure in the line 1409 is less than the pressure differential set point, the check valve 2350 closes the line 1416. In some applications, a pressure range in the line 1409 will be known. The use of a check valve 2350 as the pressure limiting device allows the pressure range in the line 1414 to be set at a predetermined level above the pressure range in the line 1409. In one exemplary embodiment, the check valve 2350 is selected to set a predetermined minimum pressure in the line 1414.

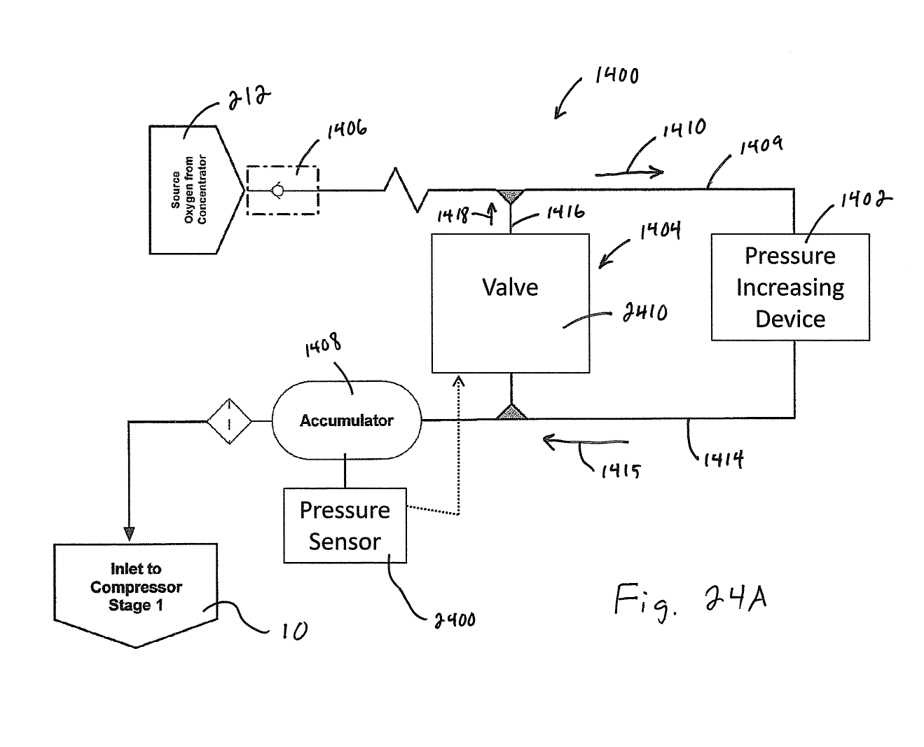

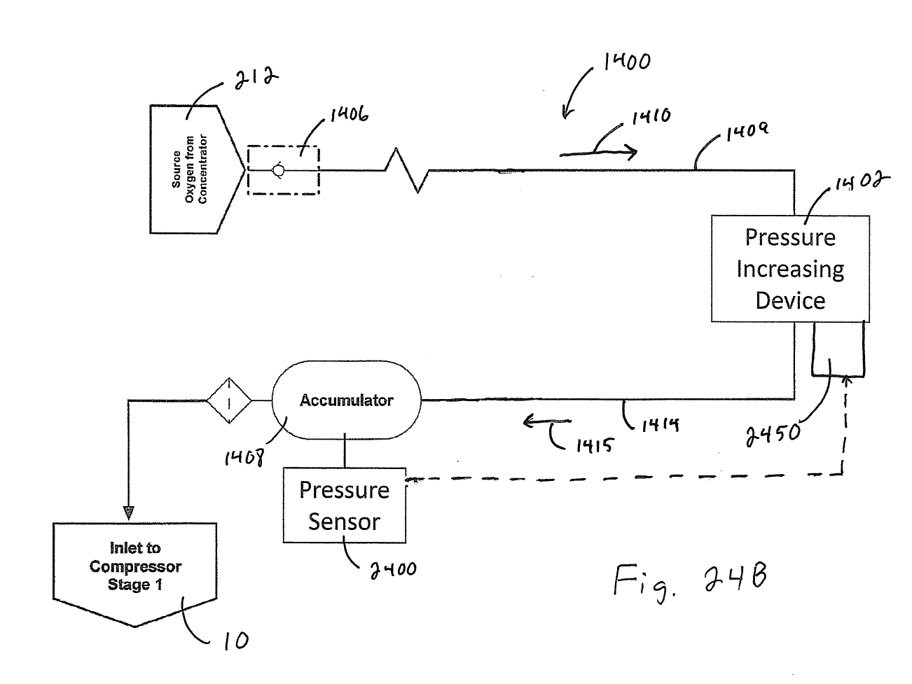

Referring to FIGS. 24A and 24B, in one exemplary embodiment, the pressure limiting device 1404 includes a pressure sensor 2400. An output of the pressure sensor 2400 may be used in a wide variety of different ways to limit the pressure of the concentrated oxygen provided to the compressor 10. In the example illustrated by FIG. 24A, the pressure limiting device 1404 includes a pressure sensor 2400 and a valve 2410 that is opened and closed based on the pressure in the line 1414 sensed by the pressure sensor 2400. When the pressure sensor 2400 senses that the pressure in the line 1414 is below the pressure set point, the valve 2410 closes the line 1416. When the pressure sensor 2400 senses that the pressure in the line 1414 is greater than the pressure set point, the valve 2410 opens the line 1416.

In the example illustrated by FIG. 24B, the pressure limiting device 1404 includes a pressure sensor 2400 and a control device 2450 that controls the pressure increasing device 1402 based on the pressure in the line 1414 sensed by the pressure sensor 2400. When the pressure sensor 2400 senses that the pressure in the line 1414 is below the pressure set point, the pressure increasing device 1402 is operated to increase the pressure in the line 1414. When the pressure sensor 2400 senses that the pressure in the line 1414 is greater than the pressure set point, the control device 2450 operates the pressure increasing device 1402 to reduce the pressure in the line 1412. For example, the control device 2450 may enable/disable and/or speed up/slow down operation of the pressure increasing device to regulate the pressure in the line 1414 at the pressure set point.

The boost stage 1400 may be used in the systems 210, 210a or any other system where concentrated oxygen is compressed by a compressor. The boost stage 1400 may be included anywhere in the fluid circuit of the systems 210, 210a between the concentrator 212 and the compressor 10. For example, boost stage may be provided immediately after the concentrator 212, immediately before the compressor 10 or anywhere in the fluid circuit between the concentrator 212 and the compressor 10. In the examples illustrated by FIGS. 12A and 13A, the boost stage 1400 is provided immediately after the concentrator 10. In this embodiment, the boost stage 1400 pre-charges the concentrated oxygen that is provided to the product tank 214 that is provided to the patient through the regulator 216, as well as the concentrated oxygen that is routed to the compressor 10.

In the examples illustrated by FIGS. 12B and 13B, the boost stage 1400 is provided after the branch between the flow path to the patient and the flow path to the compressor 10. In this embodiment, the boost stage 1400 pre-charges the concentrated oxygen that is routed to the compressor 10, but does not pre-charge the concentrated oxygen that is provided to the product tank 214 that provides concentrated oxygen to the patient through the regulator 216.

While the present invention has been illustrated by the description of embodiments thereof, and while the embodiments have been described in considerable detail, it is not the intention of the applicant to restrict or in any way limit the scope of the appended claims to such detail. Additional advantages and modifications will readily appear to those skilled in the art. Still further, while cylindrical components have been shown and described herein, other geometries can be used including elliptical, polygonal (e.g., square, rectangular, triangular, hexagonal, etc.) and other shapes can also be used. Therefore, the invention, in its broader aspects, is not limited to the specific details, the representative apparatus, and illustrative examples shown and described. Accordingly, departures can be made from such details without departing from the spirit or scope of the applicant's general inventive concept.

1. An oxygen concentration and compression system comprising:
an oxygen concentrator having an inlet that receives air and an outlet that provides a source of oxygen enriched gas at a first pressure;
a boost stage having an inlet that receives at least a portion of said oxygen enriched gas at said first pressure, a pressure increasing device that increases the pressure of the oxygen enriched gas from the first pressure to a second pressure, an outlet that provides a source of oxygen enriched gas at the second pressure, and a pressure limiting device that controls said second pressure;
a compressor having an inlet that receives at least a portion of said oxygen enriched gas at said second pressure and an outlet that provides a source of oxygen enriched gas at a third pressure that is higher than said second pressure;
a portable container that receives and stores oxygen enriched gas from the compressor at the third pressure for subsequent use by a patient;
a patient outlet that provides oxygen enriched gas from the oxygen concentrator or the boost stage for use by a patient.

2. The oxygen concentration and compression system of claim 1 wherein the pressure limiting device controls said second pressure, such that said second pressure is substantially constant.

3. The oxygen concentration and compression system of claim 1 wherein the pressure limiting device controls said second pressure, such that a difference between said second pressure and said first pressure is substantially constant.

4. The oxygen concentration and compression system of claim 1 wherein the pressure limiting device controls the second pressure by routing oxygen enriched gas from the boost stage outlet back to the boost stage inlet.

5. The oxygen concentration and compression system of claim 1 wherein the pressure limiting device controls the second pressure by a regulator that selectively feeds oxygen enriched gas from the boost stage outlet back to the boost stage inlet.

6. The oxygen concentration and compression system of claim 1 wherein the pressure limiting device comprises a pressure sensor that senses the second pressure and a valve that is controlled based on the pressure sensed by the sensor to selectively open a path from the boost stage outlet back to the boost stage inlet.

7. The oxygen concentration and compression system of claim 1 wherein the pressure limiting device comprises a pressure sensor that senses the second pressure and that controls the boost stage pressure increasing device based on the pressure sensed by the sensor.

8. The oxygen concentration and compression system of claim 1 wherein the boost stage pressure increasing device comprises a second compressor.

9. The oxygen concentration and compression system of claim 8 wherein the boost stage compressor and the compressor that increases the pressure from the second pressure to the third pressure are driven by a common motor.

10. The oxygen concentration and compression system of claim 9 wherein the boost stage compressor and the compressor that increases the pressure from the second pressure to the third pressure are driven by first and second ends, respectively, of a drive shaft of a common motor.

11. The oxygen concentration and compression system of claim 1 wherein the boost stage pressure increasing device comprises a pressure intensifier that is powered by the source of oxygen at the first pressure from the oxygen concentrator.

12. An oxygen concentration and compression system comprising:
an oxygen concentrator having an inlet that receives air and an outlet that provides a source of oxygen enriched gas at a first pressure;
a boost means for receiving at least a portion of said oxygen enriched gas at said first pressure, increasing the pressure of the oxygen enriched gas from the first pressure to a controlled second pressure, and providing a source of oxygen enriched gas at the controlled second pressure;
a compressor having an inlet that receives at least a portion of said oxygen enriched gas at said second pressure and an outlet that provides a source of oxygen enriched gas at a third pressure that is higher than said second pressure;
a portable container that receives and stores oxygen enriched gas from the compressor at the third pressure for subsequent use by a patient;
a patient outlet that provides oxygen enriched gas from the oxygen concentrator or the boost stage for use by a patient.

13. The oxygen concentration and compression system of claim 1 wherein the boost means controls said second pressure, such that said second pressure is substantially constant.

14. The oxygen concentration and compression system of claim 1 wherein the boost means controls said second pressure, such that a difference between said second pressure and said first pressure is substantially constant.

15. The oxygen concentration and compression system of claim 8 wherein the boost means and the compressor are driven by a common motor.

16. The oxygen concentration and compression system of claim 1 wherein the boost means is powered by the source of oxygen at the first pressure from the oxygen concentrator.

17. A method of concentrating oxygen and compressing the concentrated oxygen comprising:
separating oxygen from air to provide a source of oxygen enriched gas at a first pressure;
receiving at least a portion of said oxygen enriched gas at said first pressure;
increasing the pressure of the oxygen enriched gas from the first pressure to a controlled second pressure,
providing a source of oxygen enriched gas at the controlled second pressure to a compressor;
compressing the oxygen enriched gas from the second pressure to a third pressure with the compressor;
providing the oxygen enriched gas at the third pressure into a portable container for subsequent use by a patient;
providing oxygen enriched gas from the oxygen concentrator or the boost stage for use by a patient.

18. The method of claim 17 wherein the second pressure controlled to be substantially constant.

19. The method of claim 17 wherein the second pressure is controlled such that a difference between said second pressure and said first pressure is substantially constant.

20. The method of claim 17 wherein controlling the second pressure comprises routing oxygen enriched gas from at the second pressure back to a point where the pressure of the oxygen enriched gas is at the first pressure.
